(19) United States
(12) Patent Application Publication
(10) Pub. No.: US 2024/0122524 A1
(43) Pub. Date: Apr. 18, 2024

(54) Method and System for Locating Epilepsy Seizure Onset Zone and Prediction of Seizure Outcome

(71) Applicant: Carnegie Mellon University, Pittsburgh, PA (US)

(72) Inventors: Bin He, Pittsburgh, PA (US); Haiteng Jiang, HangZhou (CN)

(21) Appl. No.: 18/277,867

(22) PCT Filed: Feb. 21, 2022
(86) PCT No.: PCT/US22/17148

§ 371 Date: Aug. 18, 2023

Related U.S. Application Data
(60) Provisional application No. 63/151,322, filed on Feb. 19, 2021.

(51) Int. Cl. A61B 5/374; A61B 5/00; A61B 5/291; G16H 20/40

(52) U.S. Cl. CPC A61B 5/374; A61B 5/291; A61B 5/4094; A61B 5/7264; A61B 5/7275; A61B 5/7282; A61B 5/7435; G16H 20/40

(57) ABSTRACT

Provided is a method for locating an epilepsy seizure onset zone and prediction of seizure outcome including receiving interictal electroencephalographs from two or more points in a patient's cerebral cortex. The interictal electroencephalographs are used to determine directional information flow values which indicate dominant information flow from a non-seizure zone to a seizure onset zone. The directional informational flow values may be input into a classification model trained to predict whether the two or more points in the patient's cerebral cortex are a seizure onset zone and/or classify the patient's predicted post-treatment seizure outcome after epilepsy treatment based on the directional information flow values. An output from the classification model may indicate a location of seizure onset zone in the patient's cerebral cortex and/or the patient's predicted post-treatment seizure outcome after epilepsy treatment. Systems and computer program products are also provided.

### CROSS REFERENCE TO RELATED APPLICATIONS

This application claims the benefit of U.S. Provisional Patent Application No. 63/151,322 filed Feb. 19, 2021, which is incorporated herein by reference in its entirety.

### STATEMENT REGARDING FEDERAL FUNDING

This invention was made with government support under Grant Nos. EB021027 and NS096761, awarded by the National Institutes of Health. The government has certain rights in the invention.

### BACKGROUND

Methods of locating epilepsy seizure onset zone (SOZ) and predicting seizure outcome are provided. Systems for locating epilepsy seizure onset zone (SOZ) and predicting seizure outcome also are provided.

Epilepsy is one of the most common neurological diseases impacting about 70 million people in the world. At least one-third of epilepsy patients become drug-resistant and potential candidates for surgical resection or neuromodulation. The key to successful epilepsy surgery relies on accurate localization and safe removal of the epileptogenic zone (EZ) and an understanding of an individual patient's seizure network. An integral component for the delineation of the EZ is the SOZ: the area of cortex that initiates clinical seizures as determined predominantly by intracranial investigations. Although surgery and neuromodulation have been proven efficient in seizure reduction, the percentage of patients with unfavorable seizure outcomes leaves significant room for improvement.

Intracranial electroencephalography (iEEG), for example, stereotactic-electroencephalography (SEEG) or electrocorticography (ECoG), is a well-established neurosurgical approach to identify epileptogenic regions for intervention. iEEG uses intracranially placed electrodes to record brain activity, including, in the case of epilepsy ictal and/or interictal activity. The current gold standard of localization of epileptogenic brain regions in clinical practice depends on capturing multiple seizures during the intracranial monitoring process. The process may take multiple days, or even weeks, to complete. As such, a method or system which can evaluate a patient's epileptogenic network, estimate SOZ, and provide surgery prognoses, from analysis of brief, interictal resting-state data segments would have tremendous clinical value. Such methods and systems would vastly improve patient care and reduce medical cost.

### SUMMARY

According to some non-limiting embodiments or aspects, provided is a method for characterizing an epileptic seizure onset zone in a patient with epilepsy, the method comprising: receiving, by at least one processor, interictal electroencephalographs, optionally including interictal epileptiform discharges, from two or more points in a patient's cerebral cortex, wherein each of the two or more points represent an electrode of a plurality of electrodes. In some non-limiting embodiments or aspects, the method further comprises: determining, by the at least one processor, directional information flow between the two or more points based on the interictal encephalographs by quantifying directional information flow value(s) at each electrode of the plurality of electrodes at a plurality of frequencies, wherein the directional information flow value(s) indicate(s) information flow to an electrode from one or more other electrodes and/or from a non-seizure onset zone to a seizure onset zone. In some non-limiting embodiments or aspects, the method further comprises: inputting, by the at least one processor, the directional information flow value(s) into a classification model, wherein the classification model is trained to perform a first task and/or a second task, wherein the first task comprises locating the seizure onset zone in the patient's cerebral cortex based on the directional flow value(s), such as the inward directional flow value(s) (e.g., a summation of directional information flow from all other electrodes to a given electrode), at an electrode of the plurality of electrodes, and wherein the second task comprises classifying the patient's predicted post-treatment seizure outcome after epilepsy treatment based on the directional information flow from the non-seizure onset zone to the seizure onset zone (e.g., the averaged directional information flows from non-seizure onset zone to seizure onset zone). In some non-limiting embodiments or aspects, the method further comprises: receiving, by the at least one processor, an output from the classification model based on inputting the directional information flow value(s) into the classification model, the output indicates the location of the seizure onset zone in the patient's cerebral cortex and/or the patient's predicted post-treatment seizure outcome after epilepsy treatment.

In some non-limiting embodiments or aspects, the classification model is trained to perform the first task and the output from the classification model indicates the location of the seizure onset zone in the patient's cerebral cortex based on the directional information flow value(s). In some non-limiting embodiments or aspects, the classification model is trained to perform the second task and the output from the classification model indicates the patient's predicted post-treatment seizure outcome after epilepsy treatment based on the directional information flow value(s) from non-seizure onset zone to seizure onset zone. In some non-limiting embodiments or aspects, the directional informational flow value(s) are determined using a Granger causality analysis (GCA), and the directional information flow value(s) represent a Granger-cause. In some non-limiting embodiments or aspects, the GCA is performed using a directed transfer function (DTF). In some non-limiting embodiments or aspects, the GCA is performed using one of: a partial directed coherence (PDC), an adaptive DTF, an adaptive PDC, and/or a cross-frequency directionality.

In some non-limiting embodiments or aspects, the method further comprises: inputting 1/f power values into the classification model; and receiving a second output from the classification model based on inputting the 1/f power values into the classification model, the second output indicates the location of the seizure onset zone in the patient's cerebral cortex and/or the patient's predicted post-treatment seizure outcome after epilepsy treatment. In some non-limiting embodiments or aspects, the 1/f power values are 1/f power slopes.

In some non-limiting embodiments or aspects, the patient's predicted post-treatment seizure outcome after epilepsy treatment is classified according to a two-tier scale, an Engel outcome scale, and/or an International League Against Epilepsy (ILAE) outcome scale, the output comprises a value indicating the patient's score according to the two-tier scale, the Engel outcome scale, and/or the ILAE outcome scale, the two-tier scale comprises seizure free and non-seizure free.

In some non-limiting embodiments or aspects, the interictal electroencephalographs are obtained by one of stereotactic-electroencephalography (SEEG) or electrocorticography (ECoG). In some non-limiting embodiments or aspects, the interictal electroencephalographs are in a range of 1 Hz to 1,000 Hz. In some non-limiting embodiments or aspects, the interictal electroencephalographs range from one second to thirty minutes in duration, including any increment therebetween.

In some non-limiting embodiments or aspects, the classification model is a decision tree classification model. In some non-limiting embodiments or aspects, the decision tree classification model is a Random Forest model. In some non-limiting embodiments or aspects, the Random Forest model is a Balanced Random Forest model. In some non-limiting embodiments or aspects, the decision tree classification model is balanced by minimizing an overall cost by assigning a high cost to a misclassification of a minority class and/or either over-sampling the minority class or down-sampling a majority class, or both. In some non-limiting embodiments or aspects, the decision tree classification model is trained using a synthetic minority over-sampling (SMOTE) procedure, wherein a minority class of data is over-sampled, and wherein a majority class of data is under-sampled.

In some non-limiting embodiments or aspects, the method further comprises: determining a treatment plan for the patient based on the output from the classification model; communicating data associated with the treatment plan to a user device; and displaying the data associated with the treatment plan via a graphical user interface on the user device. In some non-limiting embodiments or aspects, the treatment plan comprises one of resective surgery, Laser interstitial thermal therapy (LITT), Responsive neurostimulation, Deep brain stimulation, Corpus callosotomy, Hemispherectomy, Functional Hemispherectomy, or no surgery.

In some non-limiting embodiments or aspects, the method further comprises: performing epilepsy surgery or laser ablation or neuromodulation on the patient when the treatment plan recommends epilepsy surgery or laser ablation or neuromodulation.

In some non-limiting embodiments or aspects, the method further comprises: categorizing the patient into a patient sub-population, the patient sub-population comprises: temporal lobe epilepsy, frontal lobe epilepsy, parietal lobe epilepsy, occipital lobe epilepsy, adult epilepsy, and pediatric epilepsy; and generating the classification model based on the patient's patient sub-population.

In some non-limiting embodiments or aspects, the method further comprises: integrating brain imaging data with the output of the classification model, the brain imaging data comprises brain images.

In some non-limiting embodiments or aspects, the method further comprises: filtering the interictal electroencephalographs using a band-pass filer between 0.5 Hz and 1,000 Hz; and performing artifact rejection on the interictal electroencephalographs.

In some non-limiting embodiments or aspects, the method further comprises: communicating data associated with the patient's predicted post-treatment seizure outcome after epilepsy treatment to a user device; and displaying the data associated with the patient's predicted post-treatment seizure outcome after epilepsy treatment via a graphical user interface on the user device.

According to non-limiting embodiments or aspects, provided is a system for characterizing an epileptic seizure onset zone in a patient with epilepsy, the system comprising at least one processor programmed or configured to: receive interictal electroencephalographs, optionally including interictal epileptiform discharges, from two or more points in a patient's cerebral cortex, wherein each of the two or more points represent an electrode of a plurality of electrodes. In some non-limiting embodiments or aspects, the at least one processor is further programmed or configured to: determine directional information flow between the two or more points based on the interictal encephalographs by quantifying the directional information flow value(s) at each electrode of the plurality of electrodes at a plurality of frequencies, wherein the directional information flow value(s) indicate(s) information flow to an electrode from one or more other electrodes and/or from a non-seizure onset zone to a seizure onset zone. In some non-limiting embodiments or aspects, the at least one processor is further programmed or configured to: input the directional information flow value(s) into a classification model, the classification model is trained to perform a first task and/or a second task, the first task comprises locating the seizure onset zone in the patient's cerebral cortex based on the directional flow value(s), such as the inward directional flow value(s), at an electrode of the plurality of electrodes, and the second task comprises classifying the patient's predicted post-treatment seizure outcome after epilepsy treatment based on the directional information flow value(s) from the non-seizure onset zone to the seizure onset zone. In some non-limiting embodiments or aspects, the at least one processor is further programmed or configured to: receive an output from the classification model based on inputting the directional information flow value(s) into the classification model, the output indicates the location of the seizure onset zone in the patient's cerebral cortex and/or the patient's predicted post-treatment seizure outcome after epilepsy treatment.

In some non-limiting embodiments or aspects, the classification model is trained to perform the first task and the output from the classification model indicates the location of the seizure onset zone in the patient's cerebral cortex based on the directional information flow value(s). In some non-limiting embodiments or aspects, the classification model is trained to perform the second task and the output from the classification model indicates the patient's predicted post-treatment seizure outcome after epilepsy treatment based on the directional information flow value(s) from non-seizure onset zone to seizure onset zone.

In some non-limiting embodiments or aspects, the directional informational flow value(s) are determined using a Granger causality analysis (GCA), and the directional information flow value(s) represent a Granger-cause. In some non-limiting embodiments or aspects, the GCA is performed using a directed transfer function (DTF). In some non-limiting embodiments or aspects, the GCA is performed using one of: a partial directed coherence (PDC), an adaptive DTF, an adaptive PDC, and/or a cross-frequency directionality.

In some non-limiting embodiments or aspects, the at least one processor is further programmed or configured to: input 1/f power values into the classification model; and receive a second output from the classification model based on inputting the 1/f power values into the classification model, the second output indicates the location of the seizure onset zone in the patient's cerebral cortex and/or the patient's predicted post-treatment seizure outcome after epilepsy treatment. In some non-limiting embodiments or aspects, the 1/f power values are 1/f power slopes.

In some non-limiting embodiments or aspects, the patient's predicted post-treatment seizure outcome after epilepsy treatment is classified according to a two-tier scale, an Engel outcome scale, and/or an International League Against Epilepsy (ILAE) outcome scale, the output comprises a value indicating the patient's score according to the two-tier scale, the Engel outcome scale, and/or the ILAE outcome scale, the two-tier scale comprises seizure free and non-seizure free.

In some non-limiting embodiments or aspects, the interictal electroencephalographs are obtained by one of stereotactic-electroencephalography (SEEG) or electrocorticography (ECoG). In some non-limiting embodiments or aspects, the interictal electroencephalographs are in a range of 1 Hz to 1,000 Hz. In some non-limiting embodiments or aspects, the interictal electroencephalographs range from one second to thirty minutes in duration, including any increment therebetween.

In some non-limiting embodiments or aspects, the classification model is a decision tree classification model. In some non-limiting embodiments or aspects, the decision tree classification model is a Random Forest model. In some non-limiting embodiments or aspects, the Random Forest model is a Balanced Random Forest model. In some non-limiting embodiments or aspects, the decision tree classification model is balanced by minimizing an overall cost by assigning a high cost to a misclassification of a minority class and/or either over-sampling the minority class or down-sampling a majority class, or both. In some non-limiting embodiments or aspects, the decision tree classification model is trained using a synthetic minority over-sampling (SMOTE) procedure, wherein a minority class of data is over-sampled, and wherein a majority class of data is under-sampled.

In some non-limiting embodiments or aspects, the at least one processor is further programmed or configured to: determine a treatment plan for the patient based on the output from the classification model; communicate data associated with the treatment plan to a user device; and display the data associated with the treatment plan via a graphical user interface on the user device. In some non-limiting embodiments or aspects, the treatment plan comprises one of resective surgery, Laser interstitial thermal therapy (LITT), Responsive neurostimulation, Deep brain stimulation, Corpus callosotomy, Hemispherectomy, Functional Hemispherectomy, or no surgery.

In some non-limiting embodiments or aspects, the at least one processor is further programmed or configured to: categorize the patient into a patient sub-population, the patient sub-population comprising: temporal lobe epilepsy, frontal lobe epilepsy, parietal lobe epilepsy, occipital lobe epilepsy, adult epilepsy, and pediatric epilepsy; and generate the classification model based on the patient's patient sub-population.

In some non-limiting embodiments or aspects, the at least one processor is further programmed or configured to: integrate brain imaging data with the output of the classification model, the brain imaging data comprises brain images.

In some non-limiting embodiments or aspects, the at least one processor is further programmed or configured to: filter the interictal electroencephalographs using a band-pass filer between 0.5 Hz and 1,000 Hz; and perform artifact rejection on the interictal electroencephalographs.

In some non-limiting embodiments or aspects, the at least one processor is further programmed or configured to: communicate data associated with the patient's predicted post-treatment seizure outcome after epilepsy treatment to a user device; and display the data associated with the patient's predicted post-treatment seizure outcome after epilepsy treatment via a graphical user interface on the user device.

According to non-limiting embodiments or aspects, provided is a computer program product for characterizing an epileptic seizure onset zone in a patient with epilepsy, the computer program product comprising at least one non-transitory computer-readable medium including one or more instructions that, when executed by at least one processor, cause the at least one processor to: receive interictal electroencephalographs, optionally including interictal epileptiform discharges, from two or more points in a patient's cerebral cortex, wherein each of the two or more points represent an electrode of a plurality of electrodes. In some non-limiting embodiments or aspects, the one or more instructions further cause the at least one processor to: determine directional information flow between the two or more points based on the interictal encephalographs by quantifying the directional information flow value(s), at each electrode of the plurality of electrodes at a plurality of frequencies, wherein the directional information flow value(s) indicate(s) information flow to an electrode from one or more other electrodes and/or from a non-seizure onset zone to a seizure onset zone. In some non-limiting embodiments or aspects, the one or more instructions further cause the at least one processor to: input the directional information flow value(s) into a classification model, the classification model is trained to perform a first task and/or a second task, the first task comprises locating the seizure onset zone in the patient's cerebral cortex based on the directional information flow value(s), such as the inward directional flow value(s), at an electrode of the plurality of electrodes, and the second task comprises classifying the patient's predicted post-treatment seizure outcome after epilepsy treatment based on the dominant directional information flow from the non-seizure onset zone to the seizure onset zone. In some non-limiting embodiments or aspects, the one or more instructions further cause the at least one processor to: receive an output from the classification model based on inputting the directional information flow value(s) into the classification model, the output indicates the location of the seizure onset zone in the patient's cerebral cortex and/or the patient's predicted post-treatment seizure outcome after epilepsy treatment.

In some non-limiting embodiments or aspects, the classification model is trained to perform the first task and the output from the classification model indicates the location of the seizure onset zone in the patient's cerebral cortex based on the directional information flow value(s). In some non-limiting embodiments or aspects, the classification model is trained to perform the second task and the output from the classification model indicates the patient's predicted post-treatment seizure outcome after epilepsy treatment based on the directional information flow value(s) from non-seizure onset zone to seizure onset zone.

In some non-limiting embodiments or aspects, the directional informational flow value(s) are determined using a Granger causality analysis (GCA), and the directional information flow value(s) represent a Granger-cause. In some non-limiting embodiments or aspects, the GCA is performed using a directed transfer function (DTF). In some non-limiting embodiments or aspects, the GCA is performed using one of: a partial directed coherence (PDC), an adaptive DTF, an adaptive PDC, and/or a cross-frequency directionality.

In some non-limiting embodiments or aspects, the one or more instructions further cause the at least one processor to: input 1/f power values into the classification model; and receive a second output from the classification model based on inputting the 1/f power values into the classification model, the second output indicates the location of the seizure onset zone in the patient's cerebral cortex and/or the patient's predicted post-treatment seizure outcome after epilepsy treatment. In some non-limiting embodiments or aspects, the 1/f power values are 1/f power slopes.

In some non-limiting embodiments or aspects, the patient's predicted post-treatment seizure outcome after epilepsy surgery is classified according to a two-tier scale, an Engel outcome scale, and/or an International League Against Epilepsy (ILAE) outcome scale, the output comprises a value indicating the patient's score according to the two-tier scale, the Engel outcome scale, and/or the ILAE outcome scale, and the two-tier scale comprises seizure free and non-seizure free.

In some non-limiting embodiments or aspects, the interictal electroencephalographs are obtained by one of stereotactic-electroencephalography (SEEG) or electrocorticography (ECoG). In some non-limiting embodiments or aspects, the interictal electroencephalographs are in a range of 1 Hz to 1,000 Hz. In some non-limiting embodiments or aspects, the interictal electroencephalographs range from one second to thirty minutes in duration, including any increment therebetween.

In some non-limiting embodiments or aspects, the classification model is a decision tree classification model. In some non-limiting embodiments or aspects, the decision tree classification model is a Random Forest model. In some non-limiting embodiments or aspects, the Random Forest model is a Balanced Random Forest model. In some non-limiting embodiments or aspects, the decision tree classification model is balanced by minimizing the overall cost by assigning a high cost to a misclassification of a minority class and/or either over-sampling the minority class or down-sampling the majority class, or both. In some non-limiting embodiments or aspects, the decision tree classification model is trained using a synthetic minority over-sampling (SMOTE) procedure, wherein a minority class of data is over-sampled, and wherein a majority class of data is under-sampled.

In some non-limiting embodiments or aspects, the one or more instructions further cause the at least one processor to: determine a treatment plan for the patient based on the output from the classification model; communicate data associated with the treatment plan to a user device; and display the data associated with the treatment plan via a graphical user interface on the user device. In some non-limiting embodiments or aspects, the treatment plan comprises one of resective surgery, Laser interstitial thermal therapy (LITT), Responsive neurostimulation, Deep brain stimulation, Corpus callosotomy, Hemispherectomy, Functional Hemispherectomy, or no surgery.

In some non-limiting embodiments or aspects, the one or more instructions further cause the at least one processor to: categorize the patient into a patient sub-population, the patient sub-population comprises: temporal lobe epilepsy, frontal lobe epilepsy, parietal lobe epilepsy, occipital lobe epilepsy, adult epilepsy, and pediatric epilepsy; and generate the classification model based on the patient's patient sub-population.

In some non-limiting embodiments or aspects, the one or more instructions further cause the at least one processor to: integrate brain imaging data with the output of the classification mode, the brain imaging data comprises brain images.

In some non-limiting embodiments or aspects, the one or more instructions further cause the at least one processor to: filter the interictal electroencephalographs using a band-pass filer between 0.5 Hz and 1,000 Hz; and perform artifact rejection on the interictal electroencephalographs.

In some non-limiting embodiments or aspects, the one or more instructions further cause the at least one processor to: communicate data associated with the patient's predicted post-treatment seizure outcome after epilepsy treatment to a user device; and display the data associated with the patient's predicted post-treatment seizure outcome after epilepsy treatment via a graphical user interface on the user device.

Other non-limiting embodiments or aspects are set forth in the following illustrative and exemplary numbered clauses:

Clause 1: A method for characterizing an epileptic seizure onset zone in a patient with epilepsy, the method comprising: receiving, by at least one processor, interictal electroencephalographs, optionally including interictal epileptiform discharges, from two or more points in a patient's cerebral cortex, wherein each of the two or more points represent an electrode of a plurality of electrodes; determining, by the at least one processor, directional information flow between the two or more points based on the interictal encephalographs by quantifying directional information flow value(s) at each electrode of the plurality of electrodes at a plurality of frequencies, wherein the directional information flow value(s) indicate(s) information flow to an electrode from one or more other electrodes and/or from a non-seizure onset zone to a seizure onset zone; inputting, by the at least one processor, the directional information flow value(s) into a classification model, wherein the classification model is trained to perform a first task and/or a second task, wherein the first task comprises locating the seizure onset zone in the patient's cerebral cortex based on the directional flow value(s), such as the inward directional flow value(s) (e.g., a summation of directional information flow from all other electrodes to a given electrode), at an electrode of the plurality of electrodes, and wherein the second task comprises classifying the patient's predicted post-treatment seizure outcome after epilepsy treatment based on the dominant directional information flow from the non-seizure onset zone to the seizure onset zone (e.g., the averaged directional information flows from non-seizure onset zone to seizure onset zone); and receiving, by the at least one processor, an output from the classification model based on inputting the directional information flow value(s) into the classification model, wherein the output indicates the location of the seizure onset zone in the patient's cerebral cortex and/or the patient's predicted post-treatment seizure outcome after epilepsy treatment.

Clause 2: The method of clause 1, wherein the classification model is trained to perform the first task and wherein the output from the classification model indicates the location of the seizure onset zone in the patient's cerebral cortex based on the directional information flow value(s).

Clause 3: The method of clause 1 or 2, wherein the classification model is trained to perform the second task and wherein the output from the classification model indicates the patient's predicted post-treatment seizure outcome after epilepsy treatment based on the directional information flow value(s) from the non-seizure onset zone to the seizure onset zone.

Clause 4: The method of any of clauses 1-3, wherein the directional informational flow value(s) are determined using a Granger causality analysis (GCA), and wherein the directional information flow value(s) represent a Granger-cause.

Clause 5: The method of any of clauses 1-4, wherein the GCA is performed using a directed transfer function (DTF).

Clause 6: The method of any of clauses 1-5, wherein the GCA is performed using one of: a partial directed coherence (PDC), an adaptive DTF, an adaptive PDC, and/or a cross-frequency directionality.

Clause 7: The method of any of clauses 1-6, further comprising: inputting 1/f power values into the classification model; and receiving a second output from the classification model based on inputting the 1/f power values into the classification model, wherein the second output indicates the location of the seizure onset zone in the patient's cerebral cortex and/or the patient's predicted post-treatment seizure outcome after epilepsy treatment.

Clause 8: The method of any of clauses 1-7, wherein the 1/f power values are 1/f power slopes.

Clause 9: The method of any of clauses 1-8, wherein the patient's predicted post-treatment seizure outcome after epilepsy treatment is classified according to a two-tier scale, an Engel outcome scale, and/or an International League Against Epilepsy (ILAE) outcome scale, wherein the output comprises a value indicating the patient's score according to the two-tier scale, the Engel outcome scale, and/or the ILAE outcome scale, wherein the two-tier scale comprises seizure free and non-seizure free.

Clause 10: The method of any of clauses 1-9, wherein the interictal electroencephalographs are obtained by one of stereotactic-electroencephalography (SEEG) or electrocorticography (ECoG).

Clause 11: The method of any of clauses 1-10, wherein the interictal electroencephalographs are in a range of 1 Hz to 1,000 Hz.

Clause 12: The method of any of clauses 1-11, wherein the interictal electroencephalographs range from one second to thirty minutes in duration, including any increment therebetween.

Clause 13: The method of any of clauses 1-12, wherein the classification model is a decision tree classification model.

Clause 14: The method of any of clauses 1-13, wherein the decision tree classification model is a Random Forest model.

Clause 15: The method of any of clauses 1-14, wherein the Random Forest model is a Balanced Random Forest model.

Clause 16: The method of any of clauses 1-15, wherein the decision tree classification model is balanced by minimizing an overall cost by assigning a high cost to a misclassification of a minority class and/or either over-sampling the minority class or down-sampling a majority class, or both.

Clause 17: The method of any of any of clauses 1-16, wherein the decision tree classification model is trained using a synthetic minority over-sampling (SMOTE) procedure, wherein the minority class of data is over-sampled, and wherein the majority class of data is under-sampled.

Clause 18: The method of any of clauses 1-17, further comprising: determining a treatment plan for the patient based on the output from the classification model; communicating data associated with the treatment plan to a user device; and displaying the data associated with the treatment plan via a graphical user interface on the user device.

Clause 19: The method of any of clauses 1-18, wherein the treatment plan comprises one of resective surgery, Laser interstitial thermal therapy (LITT), Responsive neurostimulation, Deep brain stimulation, Corpus callosotomy, Hemispherectomy, Functional Hemispherectomy, or no surgery.

Clause 20: The method of any of clauses 1-19, further comprising: performing epilepsy surgery or laser ablation or neuromodulation on the patient when the treatment plan recommends epilepsy surgery or laser ablation or neuromodulation.

Clause 21: The method of any of clauses 1-20, further comprising: categorizing the patient into a patient sub-population, wherein the patient sub-population comprises: temporal lobe epilepsy, frontal lobe epilepsy, parietal lobe epilepsy, occipital lobe epilepsy, adult epilepsy, and pediatric epilepsy; and generating the classification model based on the patient's patient sub-population.

Clause 22: The method of any of clauses 1-21, further comprising: integrating brain imaging data with the output of the classification model, wherein the brain imaging data comprises brain images.

Clause 23: The method of any of clauses 1-22, further comprising: filtering the interictal electroencephalographs using a band-pass filer between 0.5 Hz and 1,000 Hz; and performing artifact rejection on the interictal electroencephalographs.

Clause 24: The method of any of clauses 1-23, further comprising: communicating data associated with the patient's predicted post-treatment seizure outcome after epilepsy treatment to a user device; and displaying the data associated with the patient's predicted post-treatment seizure outcome after epilepsy treatment via a graphical user interface on the user device.

Clause 25: A system for characterizing an epileptic seizure onset zone in a patient with epilepsy, the system comprising at least one processor programmed or configured to: receive interictal electroencephalographs, optionally including interictal epileptiform discharges, from two or more points in a patient's cerebral cortex, wherein each of the two or more points represent an electrode of a plurality of electrodes; determine directional information flow between the two or more points based on the interictal encephalographs by quantifying the directional information flow value(s) at each electrode of the plurality of electrodes at a plurality of frequencies, wherein the directional information flow value(s) indicate(s) information flow to an electrode from one or more other electrodes and/or from a non-seizure onset zone to a seizure onset zone; input the directional information flow value(s) into a classification model, wherein the classification model is trained to perform a first task and/or a second task, wherein the first task comprises locating the seizure onset zone in the patient's cerebral cortex based on the directional flow value(s), such as the inward directional flow value(s), at an electrode of the plurality of electrodes, and wherein the second task comprises classifying the patient's predicted post-treatment seizure outcome after epilepsy treatment based on the directional information flow value(s) from the non-seizure onset zone to the seizure onset zone; and receive an output from the classification model based on inputting the directional information flow value(s) into the classification model, wherein the output indicates the location of the seizure onset zone in the patient's cerebral cortex and/or the patient's predicted post-treatment seizure outcome after epilepsy treatment.

Clause 26: The system of clause 25, wherein the classification model is trained to perform the first task and wherein the output from the classification model indicates the location of the seizure onset zone in the patient's cerebral cortex based on the directional information flow value(s), such as the inward directional flow value(s).

Clause 27: The system of clause 25 or 26, wherein the classification model is trained to perform the second task and wherein the output from the classification model indicates the patient's predicted post-treatment seizure outcome after epilepsy treatment based on the directional information flow value(s) from the non-seizure onset zone to the seizure onset zone.

Clause 28: The system of any of clauses 25-27, wherein the directional informational flow value(s) are determined using a Granger causality analysis (GCA), and wherein the directional information flow value(s) represent a Granger-cause.

Clause 29: The system of any of clauses 25-28, wherein the GCA is performed using a directed transfer function (DTF).

Clause 30: The system of any of clauses 25-29, wherein the GCA is performed using one of: a partial directed coherence (PDC), an adaptive DTF, an adaptive PDC, and/or a cross-frequency directionality.

Clause 31: The system of any of clauses 25-30, wherein the processor is further programmed or configured to: input 1/f power values into the classification model; and receive a second output from the classification model based on inputting the 1/f power values into the classification model, wherein the second output indicates the location of the seizure onset zone in the patient's cerebral cortex and/or the patient's predicted post-treatment seizure outcome after epilepsy treatment.

Clause 32: The system of any of clauses 25-31, wherein the 1/f power values are1/f power slopes.

Clause 33: The system of any of clauses 25-32, wherein the patient's predicted post-treatment seizure outcome after epilepsy treatment is classified according to a two-tier scale, an Engel outcome scale, and/or an International League Against Epilepsy (ILAE) outcome scale, wherein the output comprises a value indicating the patient's score according to the two-tier scale, the Engel outcome scale, and/or the ILAE outcome scale, wherein the two-tier scale comprises seizure free and non-seizure free.

Clause 34: The system of any of clauses 25-33, wherein the interictal electroencephalographs are obtained by one of stereotactic-electroencephalography (SEEG) or electrocorticography (ECoG).

Clause 35: The system of any of clauses 25-34, wherein the interictal electroencephalographs are in a range of 1 Hz to 1,000 Hz.

Clause 36: The system of any of clauses 25-35, wherein the interictal electroencephalographs range from one second to thirty minutes in duration, including any increment therebetween.

Clause 37: The system of any of clauses 25-36, wherein the classification model is a decision tree classification model.

Clause 38: The system of any of clauses 25-37, wherein the decision tree classification model is a Random Forest model.

Clause 39: The system of any of clauses 25-38, wherein the Random Forest model is a Balanced Random Forest model.

Clause 40: The system of any of clauses 25-39, wherein the decision tree classification model is balanced by minimizing an overall cost by assigning a high cost to a misclassification of a minority class and/or either over-sampling the minority class or down-sampling a majority class, or both.

Clause 41: The system of any of clauses 25-40, wherein the decision tree classification model is trained using a synthetic minority over-sampling (SMOTE) procedure, wherein a minority class of data is over-sampled, and wherein a majority class of data is under-sampled.

Clause 42: The system of any of clauses 25-41, wherein the at least one processor is further programmed or configured to: determine a treatment plan for the patient based on the output from the classification model; communicate data associated with the treatment plan to a user device; and display the data associated with the treatment plan via a graphical user interface on the user device.

Clause 43: The system of any of clauses 25-42, wherein the treatment plan comprises one of resective surgery, Laser interstitial thermal therapy (LITT), Responsive neurostimulation, Deep brain stimulation, Corpus callosotomy, Hemispherectomy, Functional Hemispherectomy, or no surgery.

Clause 44: The system of any of clauses 25-43, wherein the at least one processor is further programmed or configured to: categorize the patient into a patient sub-population, wherein the patient sub-population comprises: temporal lobe epilepsy, frontal lobe epilepsy, parietal lobe epilepsy, occipital lobe epilepsy, adult epilepsy, and pediatric epilepsy; and generate the classification model based on the patient's patient sub-population.

Clause 45: The system of any of clauses 25-44, wherein the at least one processor is further programmed or configured to: integrate brain imaging data with the output of the classification model, wherein the brain imaging data comprises brain images.

Clause 46: The system of any of clauses 25-45, wherein the at least one processor is further programmed or configured to: filter the interictal electroencephalographs using a band-pass filer between 0.5 Hz and 1,000 Hz; and perform artifact rejection on the interictal electroencephalographs.

Clause 47: The system of any of clauses 25-46, wherein the at least one processor is further programmed or configured to: communicate data associated with the patient's predicted post-treatment seizure outcome after epilepsy treatment to a user device; and display the data associated with the patient's predicted post-treatment seizure outcome after epilepsy treatment via a graphical user interface on the user device.

Clause 48: A computer program product for characterizing an epileptic seizure onset zone in a patient with epilepsy, the computer program product comprising at least one non-transitory computer-readable medium including one or more instructions that, when executed by at least one processor, cause the at least one processor to: receive interictal electroencephalographs, optionally including interictal epileptiform discharges, from two or more points in a patient's cerebral cortex, wherein each of the two or more points represent an electrode of a plurality of electrodes; determine directional information flow between the two or more points based on the interictal encephalographs by quantifying the directional information flow value(s), at each electrode of the plurality of electrodes at a plurality of frequencies, wherein the directional information flow value(s) indicate(s) information flow to an electrode from one or more other electrodes and/or from a non-seizure onset zone to a seizure onset zone; input the directional information flow value(s) into a classification model, wherein the classification model is trained to perform a first task and/or a second task, wherein the first task comprises locating the seizure onset zone in the patient's cerebral cortex based on the directional information flow value(s), such as the inward directional flow value(s), at an electrode of the plurality of electrodes, and wherein the second task comprises classifying the patient's predicted post-treatment seizure outcome after epilepsy treatment based on the dominant directional information flow from the non-seizure onset zone to the seizure onset zone; and receive an output from the classification model based on inputting the directional information flow value(s) into the classification model, wherein the output indicates the location of the seizure onset zone in the patient's cerebral cortex and/or the patient's predicted post-treatment seizure outcome after epilepsy treatment.

Clause 49: The computer program product of clause 48, wherein the classification model is trained to perform the first task and wherein the output from the classification model indicates the location of the seizure onset zone in the patient's cerebral cortex based on the directional information flow value(s), such as the inward directional flow value(s).

Clause 50: The computer program product of clause 48 or 49, wherein the classification model is trained to perform the second task and wherein the output from the classification model indicates the patient's predicted post-treatment seizure outcome after epilepsy treatment based on the directional information flow value(s) from the non-seizure onset zone to the seizure onset zone.

Clause 51: The computer program product of any of clauses 48-50, wherein the directional informational flow value(s) are determined using a Granger causality analysis (GCA), and wherein the directional information flow value(s) represent a Granger-cause.

Clause 52: The computer program product of any of clauses 48-51, wherein the GCA is performed using a directed transfer function (DTF).

Clause 53: The computer program product of any of clauses 48-52, wherein the GCA is performed using one of: a partial directed coherence (PDC), an adaptive DTF, an adaptive PDC, and/or a cross-frequency directionality.

Clause 54: The computer program product of any of clauses 48-53, wherein the one or more instructions further cause the at least one processor to: input 1/f power values into the classification model; and receive a second output from the classification model based on inputting the 1/f power values into the classification model, wherein the second output indicates the location of the seizure onset zone in the patient's cerebral cortex and/or the patient's predicted post-treatment seizure outcome after epilepsy treatment.

Clause 55: The computer program product of any of clauses 48-54, wherein the 1/f power values are 1/f power slopes.

Clause 56: The computer program product of any of clauses 48-55, wherein the patient's predicted post-treatment seizure outcome after epilepsy surgery is classified according to a two-tier scale, an Engel outcome scale, and/or an International League Against Epilepsy (ILAE) outcome scale, wherein the output comprises a value indicating the patient's score according to the two-tier scale, the Engel outcome scale, and/or the ILAE outcome scale, and wherein the two-tier scale comprises seizure free and non-seizure free.

Clause 57: The computer program product of any of clauses 48-56, wherein the interictal electroencephalographs are obtained by one of stereotactic-electroencephalography (SEEG) or electrocorticography (ECoG).

Clause 58: The computer program product of any of clauses 48-55, wherein the interictal electroencephalographs are in a range of 1 Hz to 1,000 Hz.

Clause 59: The computer program product of any of clauses 48-58, wherein the interictal electroencephalographs range from one second to thirty minutes in duration, including any increment therebetween.

Clause 60: The computer program product of any of clauses 48-59, wherein the classification model is a decision tree classification model.

Clause 61: The computer program product of any of clauses 48-60, wherein the decision tree classification model is a Random Forest model.

Clause 62: The computer program product of any of clauses 48-61, wherein the Random Forest model is a Balanced Random Forest model.

Clause 63: The computer program product of any of clauses 48-62, wherein the decision tree classification model is balanced by minimizing the overall cost by assigning a high cost to a misclassification of a minority class and/or either over-sampling the minority class or down-sampling the majority class, or both.

Clause 64: The computer program product of any of clauses 48-63, wherein the decision tree classification model is trained using a synthetic minority over-sampling (SMOTE) procedure, wherein a minority class of data is over-sampled, and wherein a majority class of data is under-sampled.

Clause 65: The computer program product of any of clauses 48-64, wherein the one or more instructions further cause the at least one processor to: determine a treatment plan for the patient based on the output from the classification model; communicate data associated with the treatment plan to a user device; and display the data associated with the treatment plan via a graphical user interface on the user device.

Clause 66: The computer program product of any of clauses 48-65, wherein the treatment plan comprises one of resective surgery, Laser interstitial thermal therapy (LITT), Responsive neurostimulation, Deep brain stimulation, Corpus callosotomy, Hemispherectomy, Functional Hemispherectomy, or no surgery.

Clause 67: The computer program product of any of clauses 48-66, wherein the one or more instructions further cause the at least one processor to: categorize the patient into a patient sub-population, wherein the patient sub-population comprises: temporal lobe epilepsy, frontal lobe epilepsy, parietal lobe epilepsy, occipital lobe epilepsy, adult epilepsy, and pediatric epilepsy; and generate the classification model based on the patient's patient sub-population.

Clause 68: The computer program product of any of clauses 48-67, wherein the one or more instructions further cause the at least one processor to: integrate brain imaging data with the output of the classification mode, wherein the brain imaging data comprises brain images.

Clause 69: The computer program product of any of clauses 48-68, wherein the one or more instructions further cause the at least one processor to: filter the interictal electroencephalographs using a band-pass filer between 0.5 Hz and 1,000 Hz; and perform artifact rejection on the interictal electroencephalographs.

Clause 70: The computer program product of any of clauses 48-69, wherein the one or more instructions cause the at least one processor to: communicate data associated with the patient's predicted post-treatment seizure outcome after epilepsy treatment to a user device; and display the data associated with the patient's predicted post-treatment seizure outcome after epilepsy treatment via a graphical user interface on the user device.

### BRIEF DESCRIPTION OF THE DRAWINGS

Additional advantages and details are explained in greater detail below with reference to the non-limiting exemplary embodiments that are illustrated in the accompanying figures, in which:

FIG. 1 is a diagram of a non-limiting embodiment or aspect of an environment in which systems, devices, products, apparatus, and/or methods, described herein, may be implemented according to the principles of the present disclosure;

FIG. 2 is a diagram of a non-limiting embodiment or aspect of components of one or more devices of FIG. 1;

FIG. 3 is a flow diagram for a non-limiting embodiment or aspect of a process of locating seizure onset zones and predicting seizure outcomes; and

FIG. 4 is an exemplary schematic illustration of an implementation of non-limiting embodiments or aspects of the process shown in FIG. 3; and

FIGS. 5A-5C provide exemplary graphs of within-frequency information flow by means of Directed Transfer Function analysis, as described below.

FIGS. 6A-6C provide exemplary graphs showing within-frequency information flow by means of PDC, as described below.

FIGS. 7A and 7B, provide exemplary graphs showing cross-frequency information flow (FIG. 7A), and a grand averaged CFD after the k-means clustering procedure, as described below.

FIGS. 8A and 8B provide exemplary graphs of 1/f power slope values, as described below.

FIGS. 9A-9C provide exemplary graphs of an association of information flow with post-seizure outcome, as described below.

FIGS. 10A-10C provide exemplary graphs of performances of SOZ and seizure outcome predictions at an individual level, as described below.

FIGS. 11A and 11B provide exemplary graphs of within-frequency directional information flow as a function of distance between electrodes.

FIG. 12 provides an exemplary graph of a power slope in the broadband frequency range (1-250 Hz), low-frequency range (1-30 Hz), and high-frequency range (30-250 Hz), as described below.

FIGS. 13A-13C provide exemplary graphs of a within-frequency directional information flow as a function of distance between electrodes, as described below.

FIG. 14 provides a graph of control analysis of different durations in within-frequency directional network and cross-frequency directional network, as described below.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates, and grammatical variants of those words or phrases.

For purposes of the description hereinafter, the terms “end,” “upper,” “lower,” “right,” “left,” “vertical,” “horizontal,” “top,” “bottom,” “lateral,” “longitudinal,” “inward,” “outward,” and derivatives thereof shall relate to the invention as it is oriented in the drawing figures and description herein. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects of the invention. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “comprising,” “comprise” or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim etc As used herein “another” may mean at least a second or more.

As used herein, the terms “determining,” “determine,” or “determined,” and variations thereof, may mean calculating and/or estimating a value using any useful processor-implemented method, for example, without limitation, by mathematical, statistical, and/or algorithmical methods.

As used herein, the term “system” may refer to one or more computing devices or combinations of computing devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

As used herein, the term “computing device” may refer to one or more electronic devices configured to process data. A computing device may include the necessary components to receive, process, and output data, such as a processor, a display, a memory, an input device, a network interface, and/or the like. A computing device may be a mobile device, such as a cellular phone (e.g., a smartphone), a portable computer, a wearable device (e.g., watches, glasses, lenses, clothing, and/or the like), a personal digital assistant (PDA), and/or other like devices. A computing device may also include a desktop computer or other form of non-mobile computer.

As used herein, the terms “patient” or “subject” refer to members of the animal kingdom, including, but not limited to human beings.

As used herein, the terms “treatment plan,” “medical treatment,” and “treatment” with respect to a patient, refers to taking one or more actions to improve the current and/or future condition of the patient. Medical treatment may include, but is not limited to, one or more of the following actions: administering a medication or other aid (e.g., oxygen) to the patient, modifying a level of monitoring of the patient, conducting one or more tests of the patient, conducting one or more surgical or reparative operations on the patient, providing one or more therapies or therapeutics to the patient, employing one or more medical devices for use on, in, or by the patient, modifying the position of the patient, increasing or reducing patient stimulation, modifying a diet of the patient, modifying an environment of the patient, and/or the like.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

As used herein, the term “satisfying” with respect to a threshold may include meeting and/or exceeding a threshold, which may include meeting or having a value less than a minimum-type threshold, and meeting or having a value greater than a maximum-type threshold.

As used herein, “interface” refers, in the context of programming and software modules, to the languages, codes and messages that programs or modules use to communicate with each other and to the hardware, and includes computer code or other data stored on a computer-readable medium that may be executed by a processor to facilitate the interaction between software modules. In some aspects of the methods and systems described herein, software modules, such as the variant calling module, the tumor phylogeny or modules and the machine learning modules are designed as separate software components, modules, or engines, with each requiring specific data input formats, and providing specific data output formats, and, in non-limiting examples, an interface may be used to facilitate such communication between components.

As used herein, the term “graphical user interface” or “GUI” refers to a generated display with which a user may interact, either directly or indirectly (e.g., through a keyboard, mouse, touchscreen, and/or the like).

As used herein, a “patient's predicted post-treatment seizure outcome after epilepsy treatment” refers to the patient's likelihood of experiencing seizures post epilepsy treatment (e.g., resective surgery, Laser interstitial thermal therapy (LITT), Responsive neurostimulation, Deep brain stimulation, Corpus callosotomy, Hemispherectomy, Functional Hemispherectomy, or no surgery) before the patient receives epilepsy treatment, which may be expressed as a value from a classification scale. One common, exemplary, scale used to describe a patient's post-treatment outcome from epilepsy treatment is the Engel scale. The following describes a classification according to the Engel scale: Class I indicates free of disabling seizures, Class II indicates rare disabling seizures (e.g., “almost seizure-free”), Class III indicates worthwhile improvement, and Class IV indicates not worthwhile improvement. Class I of the Engel outcome scale can be further divided into subclasses IA-ID, where IA indicates completely seizure-free since surgery; IB indicates non-disabling simple partial seizures only since surgery; IC indicates some disabling seizures after surgery, but free of disabling seizures for at least two years; and ID indicates generalized convulsions with antiepileptic drug withdrawal only. Class II can be further divided into subclasses IIA-IID, where IIA indicates initially free of disabling seizures but has rare seizures now; IIB indicates rare disabling seizures since surgery; IIC indicates more than rare disabling seizures after surgery, but rare seizures for at least 2 years; and IID indicates nocturnal seizures only. Class II can be further divided into subclasses IIIA and IIIB, where IIIA indicates worthwhile seizure reduction and IIIB indicates prolonged seizure-free intervals amounting to greater than half the follow-up period, by not less than two years. Class IV can be divided into subclasses IVA-IVC, where IVA indicates significant seizure reduction; IVB indicates no appreciable change; and IVC indicates seizures are worse.

A second exemplary scale is the International League Against Epilepsy (ILAE) scale as described in Weiser et al., Proposal for a New Classification of Outcome with Respect to Epileptic Seizures Following Epilepsy Surgery, ILAE Commission Report, 2001, 42(2), pp. 282-286. The following describes a classification according to the ILAE scale: Class 1 indicates completely seizure free and no auras; Class 2 indicates only auras and no other seizures; Class 3 indicates one to three seizure days per year and may include auras; Class 4 indicates four seizure days per year or a 50% reduction of baseline seizure days and may include auras; Class 5 indicates less than a 50% reduction of baseline seizure days and may include auras; and Class 6 indicates more than a 100% increase of baseline seizure days and may include auras.

Epilepsy surgery refers generally to a physical manipulation of a patient's brain with the overall goal of reducing epileptic seizures in the patient. Non-limiting examples of epilepsy treatment procedures include: resective surgery, laser interstitial thermal therapy (LITT), deep brain stimulation, responsive neurostimulation, corpus callosotomy, hemispherectomy, or functional hemispherectomy.

Non-limiting embodiments or aspects of the present disclosure are directed to methods, systems, and/or products for localizing an epilepsy SOZ and/or predicting a post-treatment seizure outcome after epilepsy surgery. In some non-limiting embodiments or aspects, a method for characterizing an epileptic SOZ in a patient with epilepsy may include: receiving, by at least one processor, interictal resting state intracranial electroencephalographs (we will use electroencephalographs in the following to refer to intracranial electroencephalographs), optionally including interictal epileptiform discharges, from two or more points in a patient's cerebral cortex, wherein each of the two or more points represent an electrode of a plurality of electrodes. In some non-limiting embodiments or aspects, the method may include determining, by the at least one processor, directional information flow between the two or more points based on the interictal encephalographs by quantifying the directional information flow value(s) and/or 1/f power values at each electrode of the plurality of electrodes, wherein the directional information flow values indicate information flow, e.g., a summation of information flow, from all other electrodes to the said electrode where the inward information flow is evaluated, and/or information flow from a non-seizure onset zone to a seizure onset zone, for example and without limitation, the averaged directional information flows from non-seizure onset zone to seizure onset zone. In some non-limiting embodiments or aspects, the method may include inputting the directional information flow value(s) and/or the 1/f power values into a classification model, wherein the classification model is trained to perform a first task and/or a second task, wherein the first task comprises locating the seizure onset zone in the patient's cerebral cortex based on the directional flow value(s), such as the inward directional flow value(s), at an electrode of the plurality of electrodes and/or the 1/f power values, and wherein the second task comprises classifying the patient's predicted post-treatment seizure outcome after epilepsy treatment based on the dominant directional information flow from the non-seizure onset zone to the seizure onset zone. In some non-limiting embodiments or aspects, the method may include receiving an output from the classification model based on inputting the directional information flow value(s) and/or the 1/f power values into the classification model, wherein the output indicates the location of the seizure onset zone in the patient's cerebral cortex and/or the patient's predicted post-treatment seizure outcome after epilepsy treatment (e.g., before the patient receives epilepsy treatment).

In this way, non-limiting embodiments or aspects of the preset disclosure improve the accuracy of localizing an epilepsy SOZ by using interictal intracranial electroencephalographs from two or more points in a patient's cerebral cortex which are recorded at a resting state over a short period of time (e.g., a duration of one second to thirty minutes). Further, non-limiting embodiments or aspects of the present disclosure increase the classification accuracy of an epilepsy SOZ and/or prediction of a post-treatment seizure outcome after epilepsy treatment by using the interictal intracranial electroencephalographs to determine directional information flow value(s) and/or 1/f power values and input the directional information flow value(s) and/or 1/f power values into a classification model to predict the location of the SOZ and/or a patient's post-treatment seizure outcome. Further, non-limiting embodiments or aspects of the present disclosure increase the classification accuracy of prediction of a post-treatment seizure outcome after epilepsy treatment by using the interictal intracranial electroencephalographs to determine directional information flow value(s) from the non-seizure onset zone to the seizure onset zone (e.g., the averaged directional information flows from non-seizure onset zone to seizure onset zone) and/or 1/f power values and input the directional information flow value(s) from the non-seizure onset zone to the seizure onset zone and/or 1/f power values into a classification model to predict a patient's post-treatment seizure outcome after epilepsy treatment , with the SOZ determined by other methods, including clinician determined SOZ or SOZ estimated by using other algorithms.

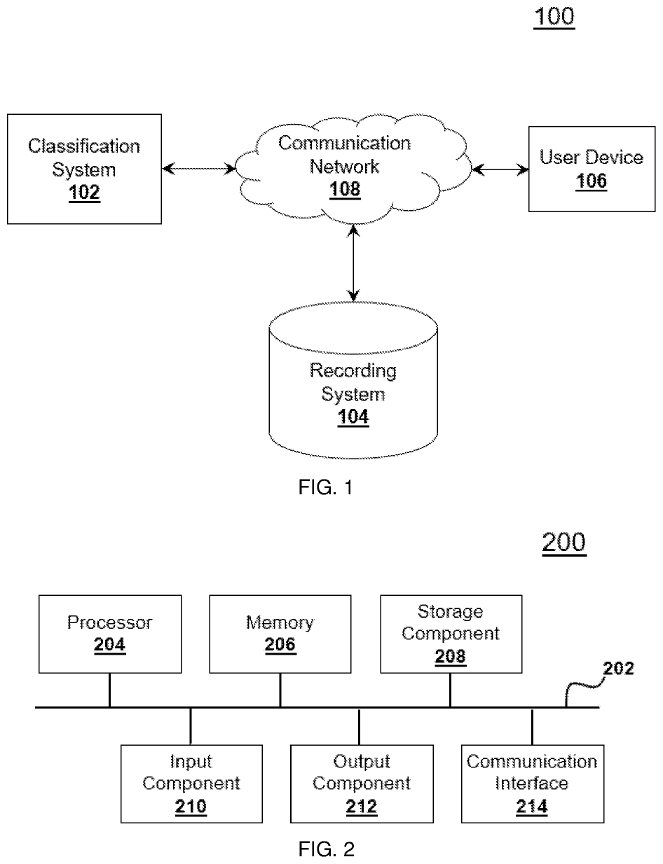

Referring now to FIG. 1, shown is a diagram of a non-limiting embodiment or aspect of an environment 100 in which devices, system, and/ or methods, described herein, may be implemented. As shown in FIG. 1, environment 100 includes classification system 102, recording system 104, user device 106, and communication network 108. Classification system 102, recording system 104, and/or user device 106 may interconnect (e.g., establish a connection to communicate) via wired connections, wireless connections, or a combination of wired and wireless connections.

Classification system 102 may include one or more devices configured to communicate with recording system 104 and/or user device 106 via communication network 108. For example, classification system 102 may include a server, a group of servers, and/or other like devices. Additionally or alternatively, classification system 102 may generate (e.g., train, validate, retrain, and/or the like), store, and/or implement (e.g., operate, provide inputs to and/or outputs from, and/or the like) one or more machine learning models. In some non-limiting embodiments or aspects, classification system 102 may be in communication with a data storage device, which may be local to (e.g., a component of) or remote from (e.g., a component of a device or system that is in communication with) classification system 102. In some non-limiting embodiments or aspects, classification system 102 may be capable of receiving information from, storing information in, transmitting information to, and/or searching information stored in the data storage device.

Recording system 104 may include one or more devices configured to communicate with classification system 102 and/or user device 106 via communication network 108. For example, recording system 104 may include a computing device, such as a server, a group of servers, and/or other like devices. In some non-limiting embodiments, recording system 104 may include one or more devices capable of producing and/or recording interictal intracranial electroencephalographs (e.g., by stereotactic-electroencephalography (SEEG) or electrococortigraphy (ECoG)), using an array of sub-dural electrodes. In some non-limiting embodiments or aspects, classification system 102 may be a component of recording system 104, or vice versa.

User device 106 may include a computing device configured to communicate with classification system 102 and/or recording system 104 via communication network 108. For example, user device 106 may include a computing device, such as a desktop computer, a portable computer (e.g., tablet computer, a laptop computer, and/or the like), a mobile device (e.g., a cellular phone, a smartphone, a personal digital assistant, a wearable device, and/or the like), and/or other like devices. In some non-limiting embodiments or aspects, user device 106 may be associated with a user (e.g., an individual operating user device 106). In some non-limiting embodiments or aspects, user device 106 may be a component of recording system 104.

Communication network 108 may include one or more wired and/or wireless networks. For example, communication network 108 may include a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN) and/or the like), a private network, an ad hoc network, an intranet, the Internet, a fiber optic-based network, a cloud computing network, and/or the like, and/or a combination of some or all of these or other types of networks.

With continued reference to FIG. 1, in some non-limiting embodiments or aspects, recording system 104 may obtain (e.g., record) interictal electroencephalographs. Interictal electroencephalographs may be obtained by any useful method, but most commonly, are obtained by intracranial electroencephalography (iEEG) methods such as SEEG or ECoG methodology and equipment. In SEEG, two or more electrodes, for example from 2 to 50 electrodes, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 electrodes, often referred to as depth electrodes, are implanted in a patient's brain, e.g., by stereotactic guidance. Each implanted electrode may have from 1-50 electrode contacts (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) electrode contacts, serving as independent point sources of iEEG data for use in the methods, systems, and software described herein. In ECoG, subdural electrode array consisting of 2˜10 by 2˜10 electrodes, such as 2 by 4 (=8) electrodes, 4 by 8 (=32 electrodes), or 8 by 8 (=64 electrodes) are placed on the surface of cortex after an open-skull operation. As such, in the methods, systems and software presented herein, iEEG data from a patient's brain, e.g., cortex, from 2 to 2,500 points, including any integer therebetween, such as data from 100 points in the patient's brain, may be simultaneously obtained and monitored. SEEG electrodes are commercially available, e.g., from DIXI Medical of Chaudefontaine, France, among other sources. Signals from the electrodes and contacts are received by a device, typically referred to as an EEG machine, which comprises suitable electronics for at least some or all of, for example and without limitation: receiving signals from the electrodes, amplifying the signals, converting analog signals to digital signals, recording the signals, filtering the signals, rejecting artifacts in the signals, analyzing the signals, and producing output corresponding to either or both of the signals or results of analyzing the signals. Output from an EEG machine receiving interictal signals may be in a form useable by the computer-implemented processes described herein, and may be analog or digital, depending on further processing conducted outside the EEG machine. EEG machines are commercially-available, but may be custom-made or adapted for the methods, systems, and software described herein.

Referring to FIG. 1, in some non-limiting embodiments or aspects, classification system 102 may receive interictal electroencephalographs, as described herein. For example, classification system 102 may receive interictal electroencephalographs, optionally including interictal epileptiform discharges, from two or more points in a patient's cerebral cortex from recording system 104. The two or more points may represent an electrode of a plurality of electrodes. In some non-limiting embodiments or aspects, the interictal electroencephalographs may be obtained by SEEG or ECoG. For example, recording system 104 may use depth or subdural electrodes to record interictal SEEG or ECoG from two or more points in a patient's cerebral cortex. In some non-limiting embodiments or aspects, the interictal electroencephalographs may range in frequency. For example, the interictal electroencephalographs may range from 1 Hz to 250 Hz, or even to 1,000 Hz. In some non-limiting embodiments or aspects, the interictal electroencephalographs may be recorded (e.g., by recording system 104) over a period of time. For example, the interictal electroencephalographs may range from one second to thirty minutes in duration, including any increment therebetween (e.g., five minutes, 10 minutes, 15 minutes, etc.).

In some non-limiting embodiments or aspects, classification system 102 may determine directional information flow between the two or more points, as described herein. For example, classification system 102 may determine directional information flow between the two or more points based on the interictal encephalographs by quantifying the directional information flow value(s) and/or 1/f power values for the points as an indication of excitation:inhibition (E:I) balance. In some non-limiting embodiments or aspects, the 1/f power value is a 1/f power slope. In some non-limiting embodiments or aspects, the directional information flow value(s) may indicate dominant information flow from a non-seizure onset zone to a seizure onset zone (SOZ). In some non-limiting embodiments or aspects, the directional information flow value(s) may be determined by Granger causality analysis (GCA) (See, e.g., Coben R, et al., Neural Connectivity in Epilepsy as Measured by Granger Causality. Front Hum Neurosci. 2015; 9:194). In some non-limiting embodiments or aspects, the directional informational flow values may represent a Granger-cause. Non-limiting examples of methods of determining Granger causality include use of directed transfer function (DTF) (see, also, e.g., He B, et al., Electrophysiological brain connectivity: Theory and implementation. IEEE Trans Biomed Eng. May 7 2019 and Kaminski M, et al., Evaluating causal relations in neural systems: Granger causality, directed transfer function and statistical assessment of significance. Biol Cybern. August 2001; 85(2):145-57); and cross-frequency directionality (CFD, see, e.g., Jiang H, et al., Multiple oscillatory push-pull antagonisms constrain seizure propagation. Ann Neurol. November 2019; 86(5):683-694 and Jiang H T, et al., Measuring directionality between neuronal oscillations of different frequencies. NeuroImage. September 2015; 118:359-367). A non-limiting example of determining a 1/f power value is 1/f power slope estimation (see, e.g., Gao R, et al., Inferring synaptic excitation/inhibition balance from field potentials. NeuroImage. September 2017; 158:70-78) are provided below. For example, the DTF analysis may be performed on the multi-electrode recordings, and the information flow at each electrode (summation of information flow from all other electrodes to the electrode of interest) at each frequency (e.g., 1 Hz, 2 Hz, 3 Hz, . . . , 250 Hz) may be calculated. The strength of such within frequency information flow may be fed into a random forest (RF) classification model to predict SOZ as described herein. In clinical applications, data greater than 250 Hz may be informative. Accordingly, in one embodiment directional information flow data may be obtained and analyzed over a range of from 1 Hz to 1,000 Hz. Other methods of Granger causality may be used, to estimate the information flow such as PDC (Partial Directed Coherence), adaptive DTF, or adaptive PDC, as are known, or using other directed functional connectivity estimation techniques.

In one aspect or embodiment, DTF may be applied to estimate the within-frequency directed information flow. For a multivariate time series Y (t) , it can be described by multivariate adaptive auto-regression (MVAR) as follows:

Y(t)=Σₖ₌₁^{p}A(k)Y(t−k)+E(t) (Eq. 1)

where A(k) is the coefficient matrix, E(t) is multivariate independent white noise, and p is the model order. The transfer function can be derived by taking the Fourier transform of Eq. (1) and then inverting the coefficient matrix, which can be described as follows:

A(f)Y(f)=E(f)

Y(f)=A⁻¹(f)E(f)=H(f)E(f) (Eq. 2)

where A(f)=Σₖ₌₀^{p}Aₖe^{-j2πfΔtk} and Aₖ₌₀=1. Here, A is the coefficient matrix obtained from the MVAR model, and H(f) is the transfer matrix, which is the inverse of the coefficient matrix and contains frequency-specific directional interaction information. The DTF value yᵢⱼ(f) represents the directional information flow from electrode i to electrode j, and is typically normalized by dividing each element by the sum of each row

$${γ}_{ij}^{2} ( f ) = \frac{{\vert {H}_{ij} ( f ) \vert}^{2}}{{Σ}_{m = 1}^{n} {\vert {H}_{im} ( f ) \vert}^{2}} \qquad ( Eq . 3 )$$

with the normalization condition Σₖ₌₁ⁿyᵢⱼ²(f)=1, where n is the number of investigated frequency bins.
For SOZ prediction: The within frequency inward information flow for electrode i, can be calculated using Eq. 3 by summing yᵢⱼ2(f) for j=all electrodes except for electrode i. This inward information flow at 1 Hz, 2Hz, . . . , 250 Hz (f in Eq. 3) can be used as inputs to the classification, e.g. decision tree or random forest model for predicting SOZ.
For Seizure outcome prediction: Perform DTF analysis of a segment of pre-processed iEEG data (SEEG or ECoG), calculate directional information flow yᵢⱼ²(f) from each non-SOZ electrode (i) to each SOZ electrode (j), obtain the strength of mean non-SOZ to SOZ within frequency information flow (e.g., 1 Hz, 2 Hz, . . . , 250 Hz) over all non-SOZ and SOZ paired electrodes for each patient can be calculated as features. Similarly, it can have 250 features (e.g., one mean value of yᵢⱼ²(f) at f=1 Hz, 2 Hz, . . . , 250 Hz) as inputs to the classification, e.g. decision tree or random forest model.

In some non-limiting embodiments or aspects, classification system 102 may input the directional information flow value(s) and/or the 1/f power values into a classification model, as described here. In some non-limiting embodiments or aspects, classification system 102 may categorize epilepsy patients into a patient sub-population. The patient sub-population may include temporal lobe epilepsy, frontal lobe epilepsy, parietal lobe epilepsy, occipital lobe epilepsy, adult epilepsy and/or pediatric epilepsy. In some non-limiting embodiments or aspects, classification system 102 may generate (e.g., train, validate, retrain, and/or the like) the classification model based on the patient's patient sub-population. In some non-limiting embodiments or aspects, the classification model may be a decision tree classification model. In some non-limiting embodiments or aspects, the classification model may be a Random Forest model. In some non-limiting embodiments or aspects, the Random Forest model may be a Balanced Random Forest model. In some non-limiting embodiments or aspects, the decision tree classification model may be balanced by minimizing an overall cost (e.g., cost-sensitive learning). (See e.g., Brownlee, Cost-Sensitive Learning for Imbalanced Classification, Machine Learning Mastery, 2020, https://machinelearningmastery.com/cost-sensitive-learning-for-imbalanced-classification/). For example, the model may assign a high cost to the misclassification of a minority class (e.g., SOZ) and/or either over-sampling the minority class, down sampling a majority class (e.g., non-SOZ), or both. In some non-limiting embodiments or aspects, the cost may be assigned automatically based on the identified number of members of each class (e.g., SOZ, non-SOZ). In some non-limiting embodiments or aspects, the decision tree classification model may be trained using a synthetic minority over-sampling (SMOTE) procedure (See, e.g., Chawla N V, et al., SMOTE: Synthetic Minority Over-sampling Technique, Journal Of Artificial Intelligence Research, 2002; 16:321-357 and Taft L M, et al. Countering imbalanced datasets to improve adverse drug event predictive models in labor and delivery, J Biomed Inform. 2009; 42(4356-364.) Using a SMOTE procedure, the minority class of data may be over-sampled and the majority class of data may be under-sampled. In some non-limiting embodiments or aspects, the classification model may be trained to perform a first task and/or a second task. In some non-limiting embodiments or aspects, the first task may include locating an SOZ in the patient's cerebral cortex based on the directional information flow value(s), such as the inward directional flow value(s), and/or the 1/f power values. In some non-limiting embodiments or aspects, the second task may include classifying the patient's predicted post-treatment seizure outcome after epilepsy treatment based on the directional information flow value(s) from the non-seizure onset zone to the seizure onset zone and/or the 1/f power values.

In some non-limiting embodiments or aspects, classification system 102 may receive an output from the classification model as described herein. For example, classification system 102 may receive an output from the classification model based on inputting the directional information flow value(s) and/or the 1/f power values into the classification model. In some non-limiting embodiments or aspects, the output may indicate the location of a SOZ in the patient's cerebral cortex and/or the patient's predicted post-treatment seizure outcome after epilepsy treatment. For example, the output may indicate the patient's predicted post-treatment seizure outcome after epilepsy treatment before the patient receives epilepsy treatment.

In some non-limiting embodiments or aspects, inputting the directional flow value(s), such as the inward directional flow value(s), and/or the 1/f power values into the classification model trained to perform the first task may result in an output from the classification model indicating a location of a SOZ in the patient's cerebral cortex based on the directional information flow value(s) and/or 1/f power values. Inputting the directional flow value(s) and/or the 1/f power values into the classification model trained to perform the second task may result in an output from the classification model indicating the patient's predicted post-treatment seizure outcome after epilepsy treatment based on the directional information flow value(s) and/or 1/f power values. In some non-limiting embodiments or aspects, the output may include a value indicating the patient's score according to a two-tier scale, an Engel outcome scale, and/or an International League Against Epilepsy (ILAE) scale. In some non-limiting embodiments or aspects, the patient's predicted post-treatment outcome after epilepsy treatment may be classified according to the two-tier scale, the Engel outcome scale and/or an ILAE scale. Other suitable scales may be used to provide a patient's predicted post-treatment seizure outcome after epilepsy treatment, and therefore may be used for classification and output in the methods and systems according to various aspects and embodiments described herein. In some non-limiting embodiments or aspects, the two-tier scale may include seizure-free and non-seizure free.

In some non-limiting embodiments or aspects, classification system 102 may determine a treatment plan for the patient based on the output from the classification model. The treatment plan may include resective surgery, laser interstitial thermal therapy (LITT), responsive neurostimulation, deep brain stimulation, corpus callosotomy, hemispherectomy, functional hemispherectomy, or no surgery (e.g., a determination that surgery is unwarranted or unlikely to yield a sufficient clinical result).

Additionally or alternatively, classification system 102 may display data associated with the indication of the location of the SOZ in the patient's cerebral cortex, data associated with the patient's predicted post-treatment outcome after epilepsy treatment, and/or data associated with the treatment plan. For example, classification system 102 may display data associated with the indication of the location of the SOZ in the patient's cerebral cortex, data associated with the patient's predicted post-treatment outcome after epilepsy treatment, and/or data associated with the treatment plan via a graphical user interface (GUI) on user device 106. In some non-limiting embodiments or aspects, the GUI may be an interactive GUI. In some non-limiting embodiments or aspects, the GUI may include at least one selectable option and/or at least one input option. The interactive GUI may be configured to be updated based on receiving at least one selection from a user and/or at least one input from a user.

The number and arrangement of systems shown in FIG. 1 are provided as an example. There may be additional systems, fewer systems, different systems, and/or differently arranged systems than those shown in FIG. 1. Furthermore, two or more systems shown in FIG. 1 may be implemented within a single system, or a single system shown in FIG. 1 may be implemented as multiple, distributed systems. Additionally, or alternatively, a set of systems (e.g., one or more systems, etc.) of environment 100 may perform one or more functions described as being performed by another set of systems of environment 100.

Referring now to FIG. 2, shown is a diagram of example components of a device 200. Device 200 may correspond to classification system 102 (e.g., one or more devices of classification system 102), recording system 104 (e.g., one or more devices of recording system 104), and/or user device 106. In some non-limiting embodiments or aspects, classification system 102, recording system 104, and/or user device 106 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage memory (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

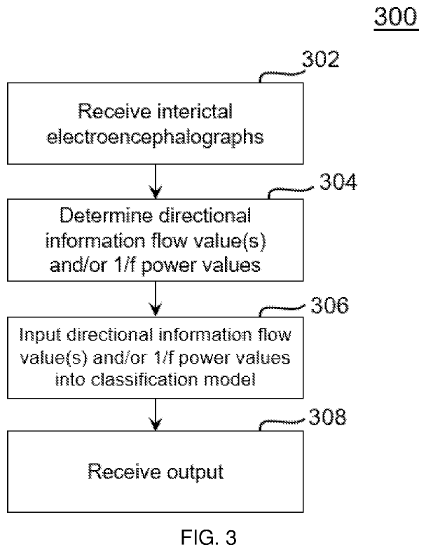

Referring now to FIG. 3, shown is a flow diagram for a non-limiting embodiment or aspect of a process 300 of locating epilepsy SOZ(s) and/or predicting post-treatment seizure outcome. The steps shown in FIG. 3 are for example purposes only. It will be appreciated that additional, fewer, different, and/or a different order of steps may be used in non-limiting embodiments. In some non-limiting embodiments or aspects, one or more steps of process 300 may be performed (e.g., completely, partially, etc.) by classification system 102 (e.g., one or more devices of classification system 102). In some non-limiting embodiments or aspects, one or more of the steps of process 300 may be performed (e.g., completely, partially, etc.) by another device or group of devices separate from or including classification system 102, such as recording system 104 (e.g., one or more devices of recording system 104), and/or user device 106 (e.g., one or more devices of user device 106).

As shown at step 302, process 300 includes receiving interictal electroencephalographs. For example, classification system 102 may receive interictal electroencephalographs from recording system 104. The interictal electroencephalographs may be from two or more points in a patient's cerebral cortex. The two or more points may represent an electrode of a plurality of electrodes in the patient's cerebral cortex. In some non-limiting embodiments or aspects, the interictal electroencephalographs may optionally include interictal elileptiform discharges.

In some non-limiting embodiments or aspects, classification system 102 may filter the interictal electroencephalographs using a band-pass filter between 0.5 Hz and 1,000 Hz. In some non-limiting embodiments or aspects, classification system 102 may perform artifact rejection on the interictal electroencephalographs. For example, as part of or immediately after receiving the interictal electroencephelographs in step 302, and prior to analysis, the interictal electroencephelographs may be filtered, and artifacts may be rejected using known methods of artifact rejection. For example, a segment of raw iEEG (e.g. a few minutes) during resting state may be selected. Data from the segment may be band-pass filtered between 0.5-1,000 Hz, and AC line noise and artifact rejected.

In some non-limiting aspects or embodiments, the preprocessed iEEG data may be selected in two ways: a) a brief (1˜10 min) segment of recordings that does not include interictal epileptiform discharges (IEDs), which shall represent resting-state information; b) a brief (1˜10 min) segment of continuous iEEG recordings which may or may not include IEDs, which shall represent interictal information.

As shown at step 304, process 300 includes determining directional information flow value(s). For example, classification system 102 may determine directional information flow between the two or more points based on the interictal encephalographs by quantifying the directional information flow in directional information flow value(s). In some non-limiting embodiments or aspects, directional information flow value(s) may indicate dominant information flow from a non-SOZ to a SOZ. In some non-limiting embodiments or aspects, the directional informational flow value(s) may be determined using a GCA, where the directional information flow value(s) represent a Granger-cause. In some non-limiting embodiments or aspects, the GCA may be performed using a DTF. Additionally or alternatively, the GCA may be performed using one of: a partial directed coherence (PDC), an adaptive DTF, an adaptive PDC, and/or a cross-frequency directionality.

Additionally or alternatively, classification system 102 may determine 1/f power values. In some non-limiting embodiments or aspects, the 1/f power values may be 1/f power slopes.

As shown at step 306, process 300 includes inputting directional information flow value(s) into a classification model. For example, classification system 102 may input directional information flow value(s) into a classification model. In some non-limiting embodiments or aspects, the classification model may be a decision tree classification model. In some non-limiting embodiments or aspects, the classification may be a Random Forest model. In some non-limiting embodiments or aspects, the classification model may be a Balanced Random Forest model. In some non-limiting embodiments or aspects, the decision tree classification model may be balanced by minimizing an overall cost by assigning a high cost to a misclassification of a minority class and/or either oversampling the minority class, down-sampling a majority class, or both. In some non-limiting embodiments or aspects, the classification model may be trained using a SMOTE procedure (e.g., over-sampling a minority class of data and under-sampling a majority class of data).

In some non-limiting embodiments or aspects, the classification model is trained to perform a first task. In some non-limiting embodiments or aspects, the first task may include locating an SOZ in the patient's cerebral cortex based on the directional information flow value(s), such as the inward directional flow value(s), and/or the 1/f power values. In some non-limiting embodiments or aspects, the classification model is trained to perform a second task. In some non-limiting embodiments or aspects, the second task may include classifying the patient's predicted post-treatment seizure outcome after epilepsy treatment based on the directional information flow value(s) and/or the 1/f power values.

Additionally or alternatively, classification system 102 may categorize the patient into a patient sub-population. The patient sub-population may include temporal lobe epilepsy, frontal lobe epilepsy, parietal lobe epilepsy, occipital lobe epilepsy, adult epilepsy, and pediatric epilepsy. In some non-limiting embodiments or aspects, classification system 102 may generate (e.g., train, validate, retrain, and/or the like) the classification model based on the patient's patient sub-population.

As shown at step 308, process 300 includes receiving an output. For example, classification system 102 may receive an output from the classification model based on inputting the directional information flow value(s) and/or the 1/f power values into the classification model. In some non-limiting embodiments or aspects, the output may indicate a SOZ in the patient's cerebral cortex and/or the patient's predicted post-treatment seizure outcome after epilepsy treatment. In some non-limiting embodiments or aspects, when the classification model is trained to perform the first task, classification system 102 may receive an output from the classification model indicating the location of the SOZ in the patient's cerebral cortex based on inputting the directional information flow value(s), such as the inward directional flow value(s), and/or 1/f power values into the classification model. In some non-limiting embodiments or aspects, when the classification model is trained to perform the second task, classification system 102 may receive an output from the classification model indicating the patient's post-treatment seizure outcome after epilepsy treatment based on inputting the directional information flow value(s) from a non-seizure onset zone to a seizure onset zone, and/or 1/f power values into the classification model. In some non-limiting embodiments or aspects, classification system 102 may integrate imaging data with the output of the classification model, where the imaging data may include brain images (e.g., magnetic resonance imaging (MRI) or computerized tomography (CT) of the brain).

Additionally or alternatively, classification system 102 may communicate data associated with the patient's predicted post-treatment seizure outcome after epilepsy treatment to user device 106. In some non-limiting embodiments or aspects, classification system 102 may displaying the data associated with the patient's predicted post-treatment seizure outcome after epilepsy treatment via a GUI on user device 106.

The classification model may be trained in any useful manner. For example, as described below, a five-fold cross validation may be used to test performance. That is, 80% of data is used to train the classification model, e.g., decision tree or random forest model, and 20% of the data is used for testing. This is repeated for a different data set, split 80% vs 20%, and performance is then averaged. In some non-limiting embodiments or aspects, the classification model may be trained in ways like the following.
A) The classification model (e.g., decision tree model, random forest model, or balanced random forest model) can be trained using data in a group of patients where the clinical ground truth is known (e.g., supervised learning). Once trained, it can be applied to data from a particular patient whose data were not included in the training dataset. For example, 10 minutes of data may be obtained during interictal stage from each of, e.g., 100 patients in whom the location of an SOZ and seizure outcome are known. The data from those 100 patients may be used to train the classification model, e.g., decision tree or random forest model. Then, for a given new patient (not part of the cohort of 100 patients for training the classification model (e.g., decision tree model, random forest model, or balanced random forest model), 10 minutes or less of interictal data can be obtained, and fed into the trained model, which will provide prediction on SOZ and/or seizure outcome.
B) The classification model (e.g., decision tree model random forest model, or balanced random forest model) can be further fine-tuned based upon patient sub-population, such as temporal lobe epilepsy, frontal lobe epilepsy, parietal lobe epilepsy, occipital lobe epilepsy, adult epilepsy group and pediatric epilepsy group. In this way, multiple classification models (e.g., decision tree models, random forest models, balanced random forest models) suitable for predicting a location of an SOZ and post-treatment seizure outcome after epilepsy treatment in specific epilepsy situation can be generated and saved, and used for an individual new patient. For example, a patient known to have temporal lobe epilepsy from other clinical information, where the exact location of the SOZ is unknown, a temporal lobe epilepsy classification model (e.g., decision tree model, random forest model, or balanced random forest model) may be used to predict the location of the SOZ using a short duration of interictal recording in this patient, and predict the post-treatment seizure outcome after epilepsy treatment, as well.

Additionally or alternatively, classification system 102 may determine a treatment plan for the patient. For example, classification system 102 may determine a treatment plan for the patient based on the output from the classification model. The treatment plan may include one of resective surgery, Laser interstitial thermal therapy (LITT), Responsive neurostimulation, Deep brain stimulation, Corpus callosotomy, Hemispherectomy, Functional Hemispherectomy, or no surgery. In some non-limiting embodiments or aspects, classification system 102 may communicate data associated with the treatment plan to user device 106. In some non-limiting embodiments or aspects, classification system 102 may display the data associated with the treatment plan via a GUI on user device 106.

Additionally or alternatively, epilepsy surgery may be performed on the patient. For example, when the treatment plan recommends epilepsy surgery, a physician may perform (e.g., manually, microscopically, robotically, etc.) epilepsy surgery on the patient, including resective surgery, Laser interstitial thermal therapy (LITT), Responsive neurostimulation, Deep brain stimulation, Corpus callosotomy, Hemispherectomy, Functional Hemispherectomy based on the treatment plan.

In a further aspect or embodiment, brain imaging data may integrated with the output of the classification system. The brain imaging data may include brain images. CT, MRI, or other brain imaging data may be co-registered with electrode location, e.g. SEEG electrode locations, to facilitate visualization of the SOZ and non-SOZ regions in a patient's brain, and to facilitate production of an output depicting or mapping, e.g., visually, holographically, or in print, the determined SOZ and/or non-SOZ areas of the patient's brain, or other aspects of the information flow or epileptogenic network of the patient's brain. Such output would inform or assist clinical diagnosis and treatment procedures, including its use in any computer-guided or robotic surgical techniques.

### EXAMPLE

A method to identify the seizure onset zone (SOZ) and predict seizure outcome using short-time resting-state iEEG (SEEG was used in this example) data was developed. In a cohort of 43 drug resistant epilepsy patients, the information flow via directional connectivity and inferred the excitation-inhibition ratio from the 1/f power slope was estimated. It was hypothesized that the antagonism of information flow at multiple frequencies between SOZ and non-SOZ underlying the relatively stable epilepsy resting state could be related to the disrupted excitation-inhibition balance. A higher excitability in non-SOZ regions compared to the SOZ, with dominant information flow from non-SOZ to SOZ regions, probably reflecting inhibitory input from non-SOZ to prevent seizure initiation was found. Greater differences in information flow between SOZ and non-SOZ regions were associated with favorable seizure outcome. By integrating a balanced random forest model with resting-state connectivity, the method localized the SOZ with an accuracy of 85% and predicted the seizure outcome with an accuracy of 77% using clinically determined SOZ. Overall, the study suggested that brief resting-state SEEG data can significantly facilitate the identification of SOZ and may eventually predict seizure outcomes without requiring long-term ictal recordings.

In a healthy state, the balanced excitation and inhibition in brain networks is regulated to facilitate information transfer and communications between remote functional regions. A number of studies have indicated that neuronal oscillations could transfer information at different frequencies, and oscillatory dysfunction has been implicated in almost every major psychiatric and neurological disorders. More specifically, it has been demonstrated that low-frequency activity (LFA, <30 Hz), high-frequency activity (HFA, >30 Hz), and LFA to HFA cross-frequency interactions of the epilepsy network are disrupted. For example, high-frequency oscillations (HFOs), interictal epileptiform discharges (IEDs), and phase-amplitude coupling (PAC) have been widely investigated as promising clinical biomarkers for epilepsy. However, HFOs, IEDs and PAC are all local biomarkers, while epilepsy is commonly accepted as a network disease. The underpinnings of seizure generation involve abnormal brain structures and aberrant functional connections among these regions, leading to large-scale network instability. Resting-state network connectivity studies have suggested predominantly increased functional connectivity involving the epileptogenic zone (EZ) and surrounding structures, and stronger inward directional connectivity toward EZ. Furthermore, decreased interictal network synchrony and local heterogeneity was found to correlate with improved seizure outcome. Therefore, a better understanding of the functional architecture of the epileptic network could help identify SOZ and improve prediction of the seizure outcome.

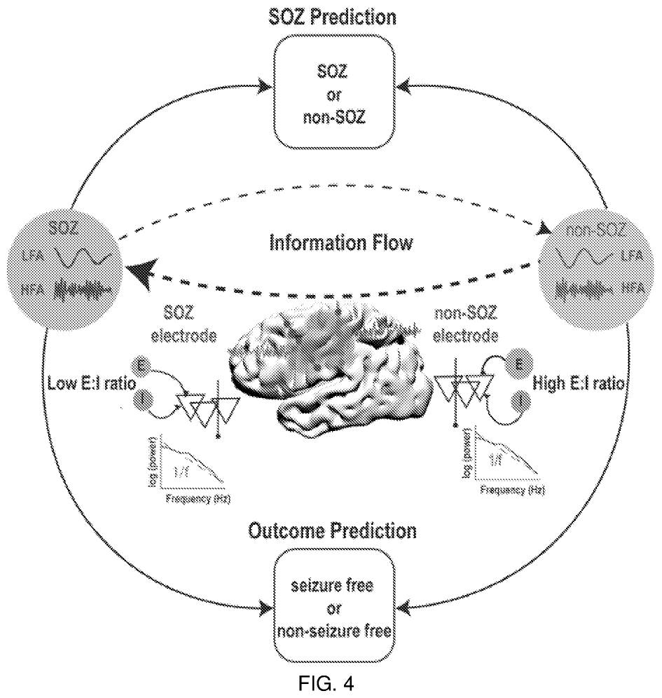

Information flow in resting-state epilepsy networks, inferred from directional connectivity in a cohort of 43 drug-resistant focal epilepsy patients was investigated. It was hypothesized that the excitation-inhibition balance is disrupted during epilepsy resting state compared to the healthy resting state and further reflected by aberrant information flow. Specifically, it was hypothesized that during the relatively stable epilepsy resting state, there are antagonisms of information flow between SOZ and non-SOZ regions at multiple frequencies. Furthermore, it was speculated that the strength of antagonisms reflects intrinsic epileptic network characteristic, which is eventually associated with seizure outcome. The goal was to develop a method to identify the SOZ for treatment intervention and to predict treatment outcomes, based on brief resting-state iEEG data without necessitating prolonged ictal recordings (FIG. 4).

In further detail, referring to FIG, 4, shown is an exemplary schematic illustration of an implementation of non-limiting embodiments or aspects of the process shown in FIG. 3. FIG. 4 is an example of a method used to identify the SOZ for treatment intervention and to predict treatment outcomes, based on brief resting-state iEEG (e.g., SEEG) data without necessitating prolonged ictal recordings. Within-frequency and cross-frequency directional connectivity (indication of information flow), 1/f power slope (indication of excitation and inhibition ratio) were investigated in the SEEG resting state data to predict SOZ and seizure outcome. LFA: Low-frequency activity; HFA: High-frequency activity; SOZ: Seizure-onset zone; E:I: Excitation:Inhibition.

### Materials and Methods

The method was tested in 43 drug-resistant focal epilepsy patients who underwent complete presurgical evaluation, including SEEG. Postoperative seizure outcome was evaluated at the last follow-up (>1 year) using the Engel classification scale (Engel J, Jr., et al. Outcome with respect to epileptic seizures. In Engel J Jr, ed. Surgical Treatment of the Epilepsies. New York; Raven Press 1993:609-621).

Ten-minute epochs were randomly selected from the long-term SEEG recordings during interictal periods in each patient when the patient was at rest. All selected epochs were recorded between 7:00 AM and 12:30 PM, and at least 2 hours away from an ictal event. The selected recordings were visually examined for the presence of epileptiform activity. If such activity was observed, the resting epoch was discarded, and another epoch was randomly selected. On average, three such epochs were obtained for each patient. Raw data were notch filtered at 60 Hz and re-referenced using bipolar montage. Electrode pairs residing in white matter were excluded from further analysis.

Information flow (directional connectivity) estimation: The within-frequency and cross-frequency directional information flow were estimated by the directed transfer function (DTF, see, e.g., He B, et al., Electrophysiological brain connectivity: Theory and implementation. IEEE Trans Biomed Eng. May 7 2019 and Kaminski M, et al., Evaluating causal relations in neural systems: Granger causality, directed transfer function and statistical assessment of significance. Biol Cybern. August 2001; 85(2):145-57) and cross-frequency directionality (CFD, see, e.g., Jiang H, et al., Multiple oscillatory push-pull antagonisms constrain seizure propagation. Ann Neurol. November 2019; 86(5):683-694 and Jiang H T, et al., Measuring directionality between neuronal oscillations of different frequencies. NeuroImage. September 2015; 118:359-367), respectively. Based on the framework of multivariate autoregressive (MVAR) models, DTF provides a spectral measure for directed information flow in the spectral domain in the multivariate system. DTF was found useful in objectively determining underlying pathological connections such as epilepsy.

Because DTF is only able to estimate the directional information flow at a single frequency, CFD is further utilized to quantify the directional interactions between different frequencies. The core basis of CFD is the phase-slope index (PSI), assuming that constant lag in the time lag could be represented by linearly increasing or decreasing phase differences in the considered frequency range. By computing the PSI between the phase of low-frequency activity (LFA) (<30 Hz) and the amplitude of high-frequency activity (HFA) (>30 Hz), the positive CFD indicates information flow from LFA to HFA and vice versa for the negative CFD.

1/f power slope estimation: The power-law exponent (slope) of the power spectrum (1/f) has been suggested to estimate synaptic excitation (E)-inhibition (I) ratios, with a flattened power slope indicating high E:I ratio and vice versa for a steep power slope (see, e.g., Gao R, et al., Inferring synaptic excitation/inhibition balance from field potentials. NeuroImage. September 2017; 158:70-78). To obtain power spectrum, data were epoched into 1-second segment without overlapping, and the time-frequency decomposition was estimated by a Fast Fourier Transformation in combination with a Hanning taper from 1 Hz to 250 Hz in 1 Hz step. Then, the power spectrum of each epoch was computed and subsequently averaged over all epochs. Essentially, the 1/f power slope is fit as a function across the selected range of the spectrum, and each oscillatory peak is modeled with a Gaussian function individually.

Random forest classification: To predict at the individual electrode level (e.g., SOZ vs. non-SOZ) and patient-level (e.g., seizure-free vs. non-seizure free outcome), the random forest machine learning technique was utilized. Random forest is an ensemble machine learning method that induces each constituent decision tree from bootstrap samples of the training data (See, e.g., Breiman L. Random forests. Machine Learning. 2001 Oct. 1 2001; 45(1):5-32). The prediction is made by aggregating all decision trees' predictions. For SOZ individual prediction, the majority of electrodes are non-SOZ. Thus, these two classes (SOZ and non-SOZ) are severely imbalanced. Dealing with highly imbalanced data, a sample may contain few or even none of the minority class, resulting in a tree with a poor predicting performance for the minority class. To tackle the problem of severely imbalanced data, a balanced random forest was introduced by adopting two strategies: 1) minimizing the overall cost by assigning a high cost to the misclassification of minority class; 2) either over-sampling the minority class or down-sampling the majority class or both. A synthetic minority over-sampling technique (SMOTE), a combination of over-sampling the minority class and under-sampling the majority class was applied (Chawla N V, et al., J Artif Intell Res. 2002; 16:321-357). To evaluate the model's performance, the five-fold cross-validation approach was applied, the receiver operator characteristic (ROC) curve was generated, and the area under the curve (AUC) was computer. The metrics of precision, recall, and overall accuracy were also assessed.

Information flow estimation using directed transfer function (DTF): The direct transfer function (DTF) was applied to estimate the within-frequency directed information flow. For a multivariate time series Y(t), it can be described by multivariate adaptive auto-regression (MVAR) as follows:

Y(t)=Σₖ₌₁^{p}A(k)Y(t−k)+E(t) (Eq. 1)

where A(k) is the coefficient matrix, E(t) is multivariate independent white noise, and p is the model order. The transfer function was derived by taking the Fourier transform of Eq. (S-1) and then inverting the coefficient matrix, which can be described as follows:

A(f)Y(f)=E(f)

Y(f)=A⁻¹(f)E(f)=H(f)E(f) (Eq. 2)

where A(f)=Σₖ₌₀^{p}Aₖe^{-j2πfΔtk} and Aₖ₌₀=I. Here, A is the coefficient matrix obtained from the MVAR model, and H(f) is the transfer matrix, which is the inverse of the coefficient matrix and contains frequency-specific directional interaction information. The DTF value yᵢᵢ(f) represents the information flow from electrode i to electrode j, and is typically normalized by dividing each element by the sum of each row

$${γ}_{ij}^{2} ( f ) = \frac{{\vert {H}_{ij} ( f ) \vert}^{2}}{{Σ}_{m = 1}^{n} {\vert {H}_{im} ( f ) \vert}^{2}} \qquad ( Eq . 3 )$$

with the normalization condition Σₖ₌₁ⁿyᵢⱼ²(f)=1, where n is the number of investigated frequency bins. From Eq. 3, the inward information flow at an electrode (sum all information flow from other electrodes toward the electrode, where the inward information flow is evaluated), and outward information flow at an electrode (sum all information flow from the electrode toward other electrodes) were calculated.

Cross-frequency directionality analysis: Cross-frequency directionality (CFD) can be used to evaluate directional interactions between different frequencies by computing the phase-slope index (PSI) between the phase of LFA and the amplitude envelope of HFA. Let x^{s} denote the raw signal at segment s and y^{v,s} denote the amplitude envelope at high frequency v. X^{s} and Y^{v,s} are defined as the Fourier transforms of x^{s} and y^{v,s}, respectively, and ξ^{v,s}=X^{s}Y̅^̅{̅v̅,̅s̅}̅ is the cross-spectrum between X^{s} and Y^{v,s}, where ‘-’ defines the complex conjugate. The complex coherency is defined as

$$C ( v , f ) = \frac{{Σ}_{s = 1}^{S} {x}^{s} ( f ) {( {Y}^{ν , s} ( f ) )}^{*}}{\sqrt{{Σ}_{s = 1}^{S} {\vert {X}^{s} ( f ) \vert}^{2} {Σ}_{s = 1}^{S} {\vert {Y}^{ν , s} ( f ) \vert}^{2}}} \qquad ( Eq . 4 )$$

The CFD between signal x and the amplitude envelope of the signal y^{v} at frequency tile (v, f) is defined as:

$$Ψ ( v , f ) = Im ( {Σ}_{g ∈ ℱ} {C ( v , g )}^{*} C ( v , g + Δ f ) ) with ℱ = \{ f - \frac{β}{2} , f - \frac{β}{2} + Δ f , … , f + \frac{β}{2} - Δ f \} \qquad ( Eq . 5 )$$

where Δf is the frequency resolution, β is the bandwidth used to calculate the phase slope and Im denotes the imaginary part.

For the CFD calculations, the high-frequency amplitude envelope was extracted using a sliding time window approach by applying a discrete Fourier transform to successive data segments after applying a Hanning taper (5 cycles long concerning the frequency of interest). This was done from 20 to 250 Hz in steps of 5 Hz. The bandwidth β for estimating the PSI was set to 2 Hz surrounding the central phase frequencies of 2 to 20 Hz in 1 Hz steps to calculate CFD.

1/f power slope estimation: The power slope was estimated using a parameterization method. This algorithm models the power spectral density (PSD) as a combination of periodic and aperiodic components, which can be described as follows:

PSD=L+Σₙ₌₀^{N}Gₙ (Eq. 6)

where L is the 1/f aperiodic component, Gₙ is a Gaussian to model each periodic component, and N is the total number of periodic components. The aperiodic component L is further modeled using a Lorentzian function as follows:

L=b−log(k+F^{λ}) (Eq. 7)

where b is the offset, F is the vector of input frequencies, χ is the exponent, and k is the ‘knee’ parameter, controlling for the bend in the aperiodic component. When there is no knee, there is a direct relationship between the slope, a of the line in log-log spacing, and the exponent χ, which χ=−a. To account for the effects of outliers on linear fitting and have an unbiased estimation, the algorithm introduces a two-step strategy by finding an initial fit of the aperiodic component and identifying only the data points along the frequency axis that are most likely not to be part of periodic components. A second fit of the original PSD is then performed only on these frequency points, giving a more robust estimation of the aperiodic component.

### Results

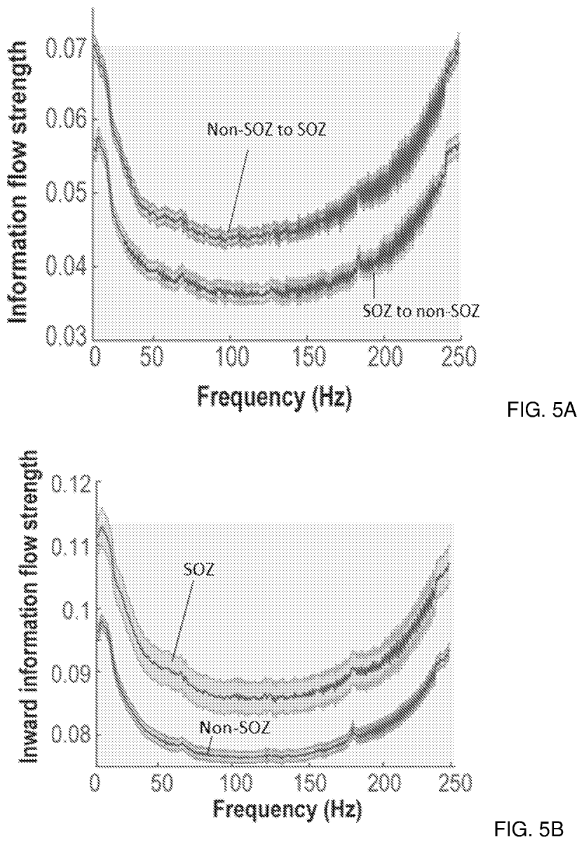

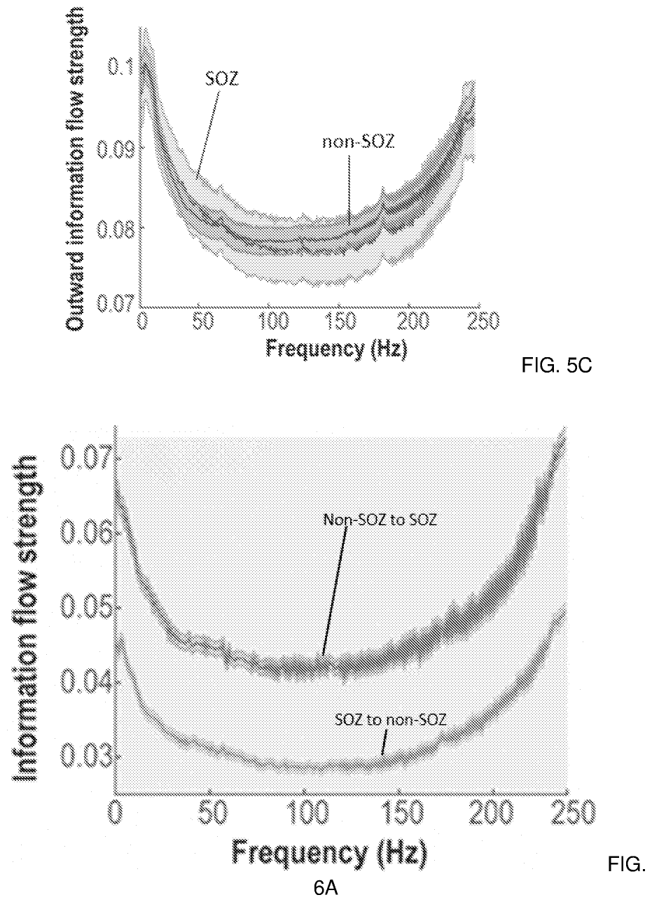

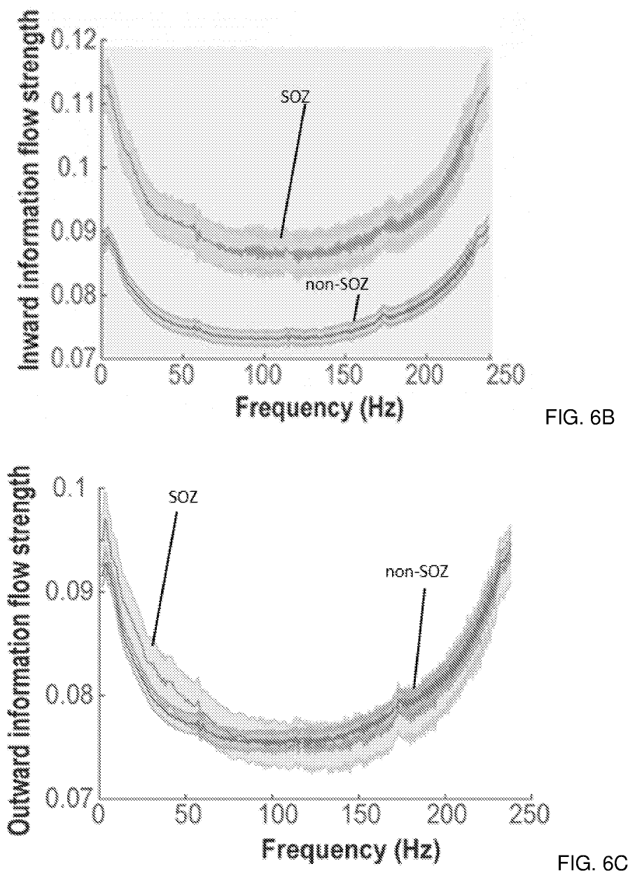

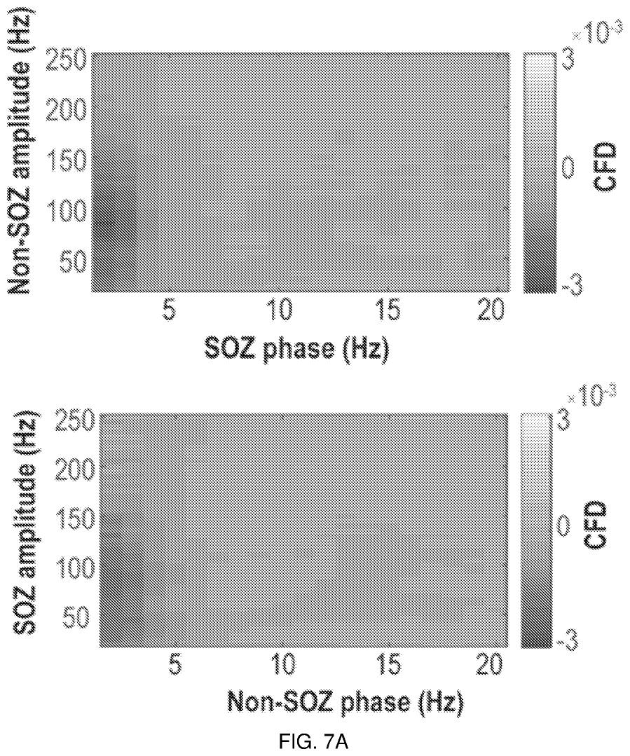

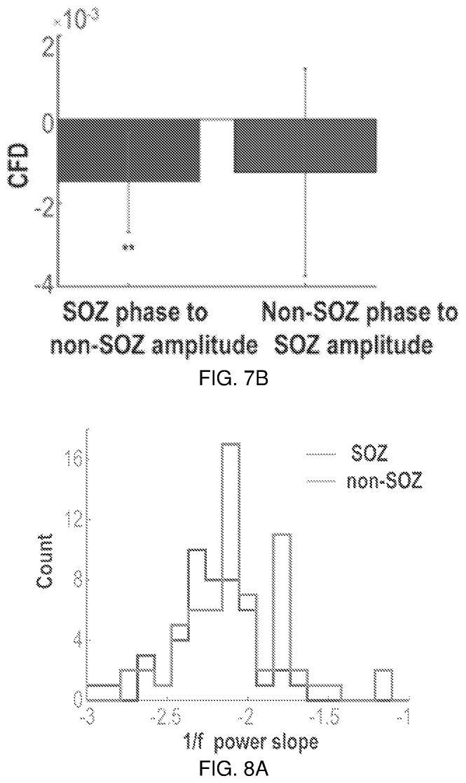

Dominant information flow from non-SOZ to SOZ underlying the resting-state: First, the confrontations of information flows between SOZ and non-SOZ were examined by comparing their differences in directional interaction both within-frequency and cross-frequency. The within-frequency directional information flow was calculated by DTF, while the cross-frequency directional information was estimated by CFD. As shown in FIG. 5A, measures of within-frequency information flow strength were significantly weaker from SOZ to non-SOZ than in the other direction, over the wide frequency range (1˜250 Hz). Furthermore, it was observed that SOZ exhibited significantly higher inward strength (mean information received from other electrodes) than non-SOZ (FIG. 5B). Patterns were similar and robust when the within frequency directional interaction was estimated by partial directed coherence (PDC) (FIG. 6), which is another technique used to estimate information flow. The results indicated that using PDC, similar to results using DTF, stronger non-SOZ to SOZ information flow and stronger inward information flow for SOZ were obtained. In the cross-frequency directional interactions, both the SOZ phase to non-SOZ amplitude and non-SOZ phase to SOZ amplitude CFD showed prominent negative 1-4 Hz to 40-150 Hz CFD (FIG. 7A), indicating information flow from HFA to LFA. Since CFD varied across electrode pairs to a different extent, k-means clustering was utilized to extract the most consistent and strongest CFD pattern across all electrode pairs (Jiang H, et al. Ann Neurol. November 2019; 86(5):683-694). After applying the k-means procedure in each patient, the significant negative CFD from SOZ phase to non-SOZ amplitude CFD (FIG. 7B) was found, suggesting the dominant cross-frequency information flow from non-SOZ HFA to SOZ LFA. Overall, the within-frequency and cross-frequency results indicated that the prevailing information flow is always from non-SOZ to SOZ.

In further detail, referring now to FIGS. 5A-5C, shown are exemplary graphs of within-frequency information flow by means of Directed Transfer Function analysis. In FIGS. 5A-5C the data is shown in mean and standard error. FIG. 5A shows mean information flows between SOZ and non-SOZ across all electrode pairs and patients. As shown in FIG. 5A, the shaded area indicates significant differences at the p=0.01 level after multiple corrections. FIG. 5B shows an exemplary inward (receiving) information flow strength in SOZ and non-SOZ. As shown in FIG. 5B, the shaded gray area indicates significant differences at the p=0.01 level after multiple corrections. FIG. 5C shows an exemplary outward (sending) information flow strength in SOZ and non-SOZ.

Referring now to FIGS. 6A-6C, shown is a within-frequency information flow by means of PDC. FIG. 6A shows the mean bidirectional information between SOZ and non-SOZ across all electrode pairs and patients. The shaded gray area indicates significant differences at the p=0.01 level after multiple comparisons. FIG. 6B shows the inward (receiving) information flow strength averaged in SOZ and non-SOZ areas. The shaded gray area indicates significant differences between SOZ and non-SOZ at the p=0.01 level after multiple comparisons. FIG. 6C shows the outward (sending) information flow strength in SOZ and non-SOZ.

Referring now to FIG. 7A, shown is an exemplary graph of a cross-frequency information flow. In FIG. 7A, the top panel shows the grand averaged SOZ phase to non-SOZ amplitude CFD across all electrode pairs and patients. In FIG. 7A, the bottom panel shown the non-SOZ phase to SOZ amplitude CFD across all electrode pairs and patients.

Referring now to FIG. 7B, shown is an exemplary graph of a grand averaged CFD after the k-means clustering procedure. As shown in FIG. 7B, the SOZ phase to non-SOZ amplitude CFD is significant compared to zero. The error bar represents standard deviation. **p<0.01.

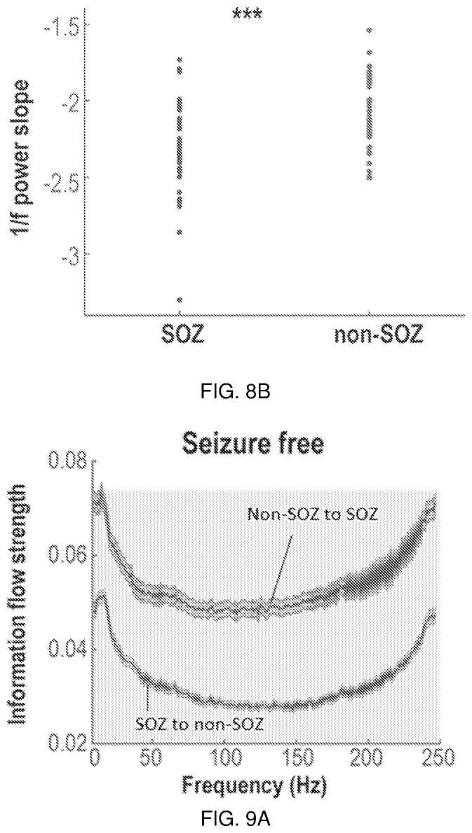

Higher excitability in non-SOZ versus SOZ revealed by 1/f power slope: The differences in information flow between SOZ and non-SOZ could be related to alternation in excitation/inhibition balance. Based on computational modeling, it has been shown that E:I ratio could be estimated from the 1/f power slope, in which the more negative power slope is associated with less excitability (Gao R, et al., NeuroImage. September 2017; 158:70-78). Therefore, the 1/f power slope as an indicator of E:I balance was investigated. After computing the power spectrum between 1 and 250 Hz at each electrode, the 1/f power slope was derived. Among 184 SOZ and 899 non-SOZ electrodes, it was found that power slopes of SOZ were significantly more negative than non-SOZ electrodes (two-sample t-test, p<10⁻⁹, FIG. 8A). On a single patient basis, 33 out of 43 patients had a more negative power slope in SOZ (FIG. 8B). Taken together, SOZ had a more negative 1/f power slope in comparison to non-SOZ, probably reflecting the higher excitability in non-SOZ.

In further detail, referring to FIGS. 8A and 8B, shown are exemplary graphs of 1/f power slope values. As shown in FIG. 8A, the distribution of 1/f power slope values shifts leftward (more negative) in SOZ vs. non-SOZ electrodes. FIG. 8B illustrates an exemplary individual-patient comparison of averaged 1/f power slopes between SOZ and non-SOZ. As shown in FIG. 8B, when SOZ and non-SOZ dots for each patient are connected by lines, each patient represented by a pair of connected dots showing the majority of patients (76.7%) are seen to have had more negative slopes in SOZ compared to non-SOZ. ***p<0.001.

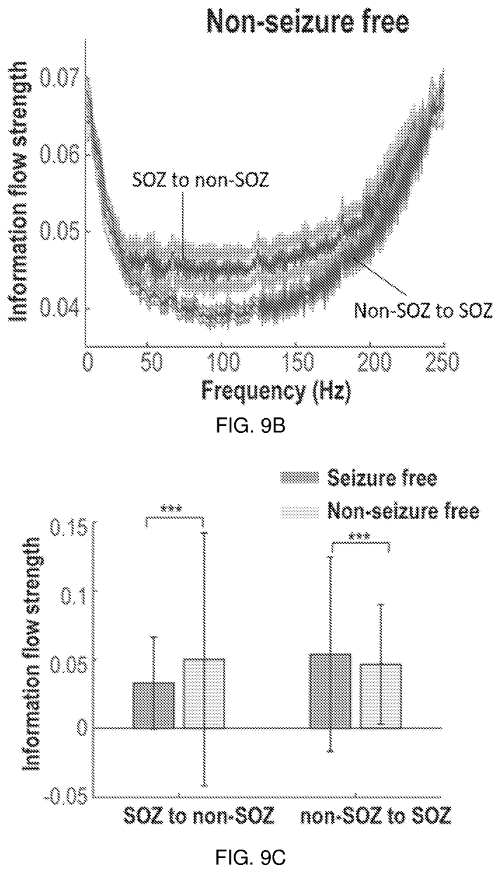

Larger information flow asymmetry between SOZ and non-SOZ is associated with favorable seizure outcome: Next, the association between connectivity and seizure outcome was investigated. Of these 43 patients, there were 22 patients with Engel I outcome (51.2%), 8 patients with Engel II outcome (18.6%), 8 patients with Engel III outcome (18.6%), and 5 patients with Engel IV outcome (11.6%). The Engel I outcome was classified as seizure-free and Engel II-IV outcome was classified as the non-seizure free outcome. Notably, the majority of seizure free outcome patients had resection (14 patients) and ablation (5 patients) treatments. In the neural data, significant differences in within-frequency information flow between SOZ and non-SOZ in the broadband frequency range in the seizure-free patients were found (FIG. 9A). At the same time, there was no significant difference in non-seizure free patients. After averaging over the broadband frequencies, the differences in seizure outcome were driven by weaker SOZ to non-SOZ information flow strength and stronger non-SOZ to SOZ information flow strength in seizure-free patients (FIG. 9C). Note that significant differences in the cross-frequency information flow were not found. Taken together, these suggested that larger within-frequency information flow asymmetry between SOZ and non-SOZ was associated with favorable seizure outcome.

In further detail, referring now to FIGS. 9A-9C, shown are exemplary graphs of an association of information flow with post-seizure outcome. FIG. 9A shows an exemplary within-frequency information flow between SOZ and non-SOZ according to seizure outcome for the patients who were seizure free. As shown in FIG. 9A, the shaded gray area indicates significant difference at the p=0.05 level after FDR correction. FIG. 9B shows an exemplary within-frequency information flow between SOZ and non-SOZ according to seizure outcome for the patients who were non-seizure free. FIG. 9C shows an exemplary averaged bidirectional within-frequency information flow between SOZ and non-SOZ over the broadband frequencies in FIG. 9A and FIG. 9B. As shown in FIG. 9C, SOZ to non-SOZ information flow strength was significantly weaker, but non-SOZ to SOZ information flow strength was substantially stronger in seizure-free than non-seizure free patients. As shown in FIG. 9C, the error bar represents standard deviation. ***p<0.001.

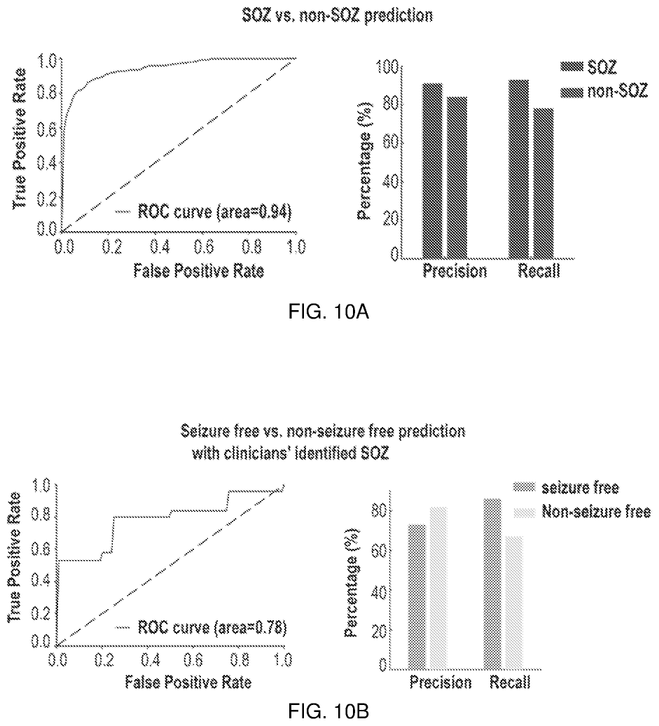

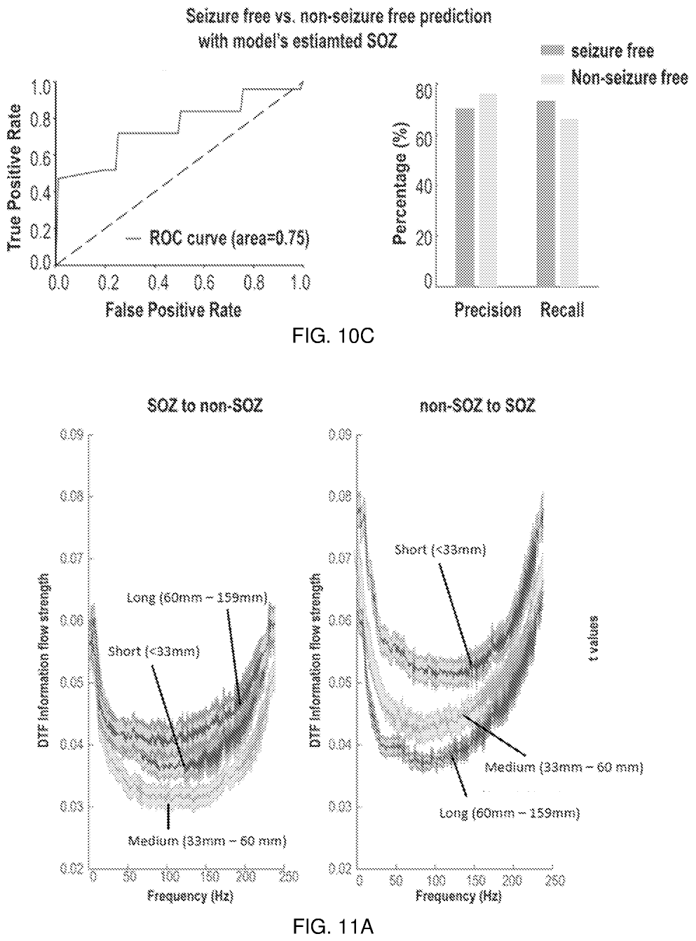

Individual predictions of SOZ and seizure outcome with random forest classifier: Lastly, the random forest classifier was used to predict: 1) whether an individual electrode is likely to be SOZ; 2) whether the patient will be seizure-free. Based on the statistical results above, within-frequency interaction was significantly different in both information flow between SOZ and non-SOZ comparison (FIGS. 5A-5C) and seizure-free outcome versus non-seizure free outcome comparison (FIGS. 9A-9C). Only the broadband within-frequency information flow was used as feature inputs into the random forest classifier to increase interpretability. More specifically, the strength of within-frequency inward information flow at each electrode was the feature for SOZ prediction, while the strength of mean non-SOZ to SOZ information flow for each patient was the feature for seizure outcome prediction. As shown in FIG. 10A, the model demonstrated an accuracy of 0.85 and an AUC of 0.94 in predicting SOZ (Precision: 0.91; Recall: 0.93) vs non-SOZ (Precision: 0.84; Recall: 0.78). For seizure outcome prediction, the model achieved an accuracy of 0.77 and an AUC of 0.78 when SOZ was identified by clinicians (Precision: [0.73 0.82]; Recall: [0.86 0.67] for seizure free and non-seizure free outcomes) (FIG. 10B). If SOZ was estimated by the prediction model, the seizure outcome prediction accuracy was 0.72 with an AUC of 0.75 (Precision: [0.71 0.77]; Recall: [0.74 0.67] for seizure free and non-seizure free outcomes) (FIG. 10C). Overall, these findings suggest that the combination of random forest and resting-state connectivity may help identify SOZ and predict seizure outcome at the individual level with satisfactory accuracy.

In further detail, referring now to FIGS. 10A-10C, shown are exemplary graphs of performances of SOZ and seizure outcome predictions at an individual level. FIG. 10A shows an example of an SOZ versus non-SOZ prediction. As shown in FIG. 10A, the receiver-operating characteristic (ROC) curves show the true-positive and false-positive rates in predicting SOZ vs. non-SOZ. The area under the curve (AUC) is 0.94. Precision=True Positive/(True Positive+False Positive); Recall=True Positive/(True Positive+False Negative); FIG. 10B shows the prediction of seizure outcome (i.e., seizure-free versus non-seizure free) with clinicians' identified SOZ. FIG. 10C shows the prediction of seizure outcome (i.e., seizure-free versus non-seizure free) with the classification model's estimated SOZ, where only 10-min resting state SEEG data were used.

Overall, the results suggest that the dominant information flow is always from non-SOZ to SOZ at multiple frequencies in the interictal period, which is probably due to the higher excitability in non-SOZ regions. Moreover, larger information flow asymmetry between SOZ and non-SOZ is associated with favorable seizure outcome. By incorporating both resting-state connectivity and random forest classifier, it is possible to localize SOZ and predict seizure outcome at the individual level with satisfactory accuracy.

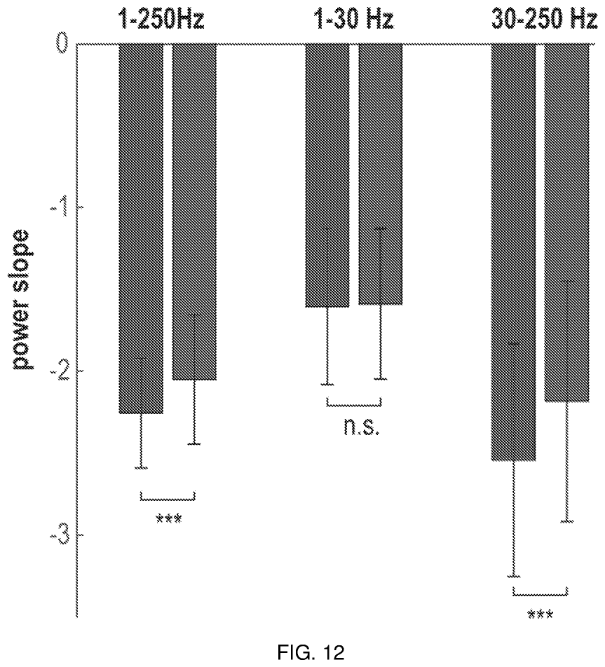

Disrupted excitation-inhibition balance in epilepsy: Neural circuits rely on a dynamic E:I balance, and the balance of E:I interaction is critical for neuronal homeostasis and neural oscillation formation. Emerging evidence indicates that E:I balance has dynamically fluctuated with neural computation, task demands, and cognitive states. More dramatic changes and aberrant E:I patterns are implicated in neurological disorders such as epilepsy. The computation model developed by Gao et al. suggested that the E:I ratio can be quantified from the power spectrum, with a flatter 1/f power slope (more positive value) indicating a higher E:I ratio (Wilke C, et al. Neocortical seizure foci localization by means of a directed transfer function method. Epilepsia. April 2010; 51(4):564-72). This was supported by the evidence that 1/f power slope tracked the propofol-induced global inhibition, in which significant slope decrease was observed during anesthesia when compared to awake. The data showed a more negative power slope in SOZ compared to non-SOZ (FIGS. 8A and 8B), probably reflecting low excitability in SOZ but high excitability in non-SOZ during the resting state. The disrupted excitation-inhibition balance might be linked to the findings in information flow. The non-SOZ with high excitability could be the source of information sender to SOZ with low excitability, explaining why the dominant information is from non-SOZ to SOZ. Moreover, a more negative power slope in SOZ was mainly driven by the high-frequency activity (30-250 Hz, FIG. 12), suggesting its frequency-specific effect.

In further detail, referring to FIG. 12, shown is an exemplary graph of a power slope in the broadband frequency range (1-250 Hz), low-frequency range (1-30 Hz), and high-frequency range (30-250 Hz). As shown in FIG. 12, there are significant differences in power slope between SOZ (left bar for each frequency range) and non-SOZ (right bar for each frequency range) in the broadband frequency range and high-frequency range but not in the low-frequency range. As shown in FIG. 12, the error bar represents standard deviation. ***p<0.001; n.s.=not significant.

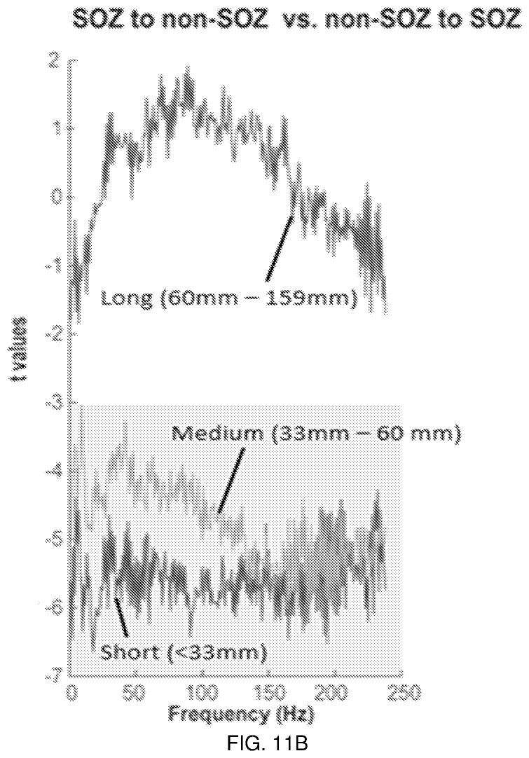

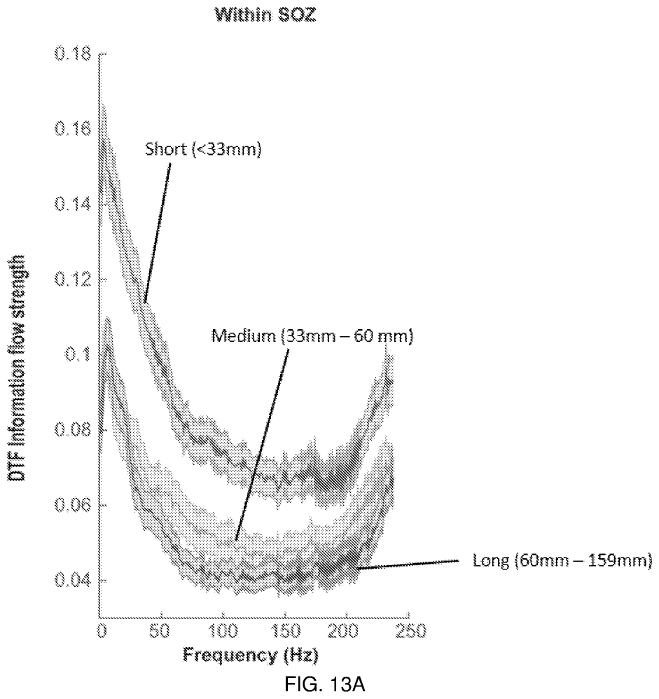

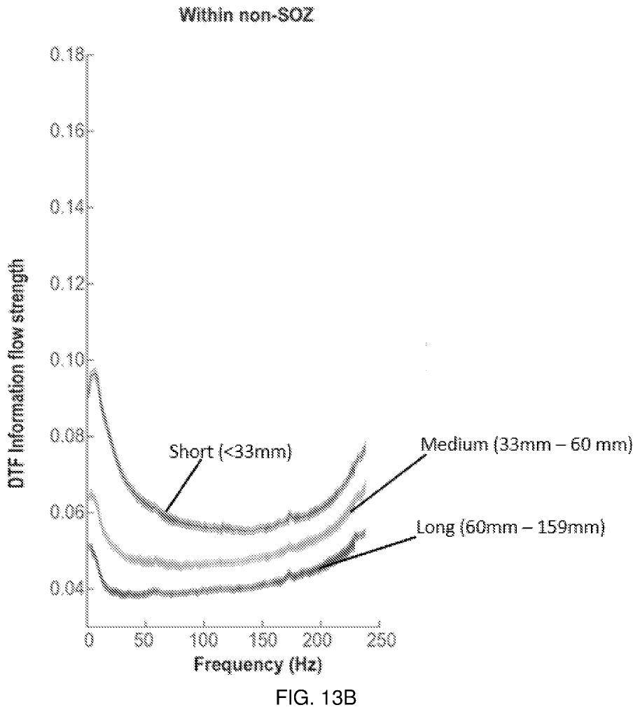

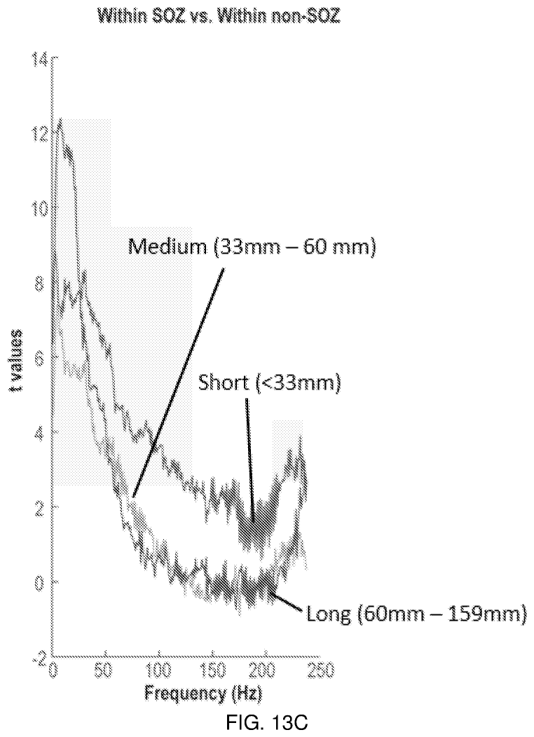

Long-range communication disruption of high-frequency activity in the epilepsy network: Synchronization between neuronal populations is critical for information transfer between brain areas. Theoretical and experimental evidence has shown that synchronization between neuronal oscillations depend on the axonal conduction delays, so LFA are generally more stably synchronized over long distance than HFA . Surprisingly, our data challenged this classical view and demonstrated HFA exhibiting long-range communication both within SOZ, within non-SOZ (FIG. 13), and between SOZ and non-SOZ (FIGS. 11A and 11B). t was estimated the information flow between all SEEG electrode pairs from 1 to 250 Hz and divided them into three groups based on distances. The information flows were first averaged over all patients in three quartiles of inter-electrode distances. The mean information flows increased from 1 to 6 Hz in all distance quartiles and then decayed to 80 Hz. However, throughout the 80-250 Hz HFA, inter-electrode DTFs started to increase again and exhibited a peak at around 240 Hz. Note that the long-range communication coordinated by HFA was shown in a recent study in healthy regions (See e.g., Arnulfo, et al., Long-range phase synchronization of high-frequency oscillations in human cortex. Nat Commun 11, 5363 (2020)). Here, the previous findings were extended and provided the first evidence of long-range communication disruption of HFA between SOZ and non-SOZ in the pathological epilepsy network. The long-range neuronal communication of HFA could arise in large-scale network. This is probably because the joint roles of local synchronization and high collective firing rates enable local pyramidal cell populations with largely increased efficiency in regulating their post-synaptic targets in distant regions. Moreover, the significant differences between SOZ and non-SOZ directional connectivity strength were found in short-range (<33 mm) and medium-range (between 33 mm and 60 mm) distance, but not in the long-range (between 60 mm and 159 mm) distance (FIGS. 11A and 11B), suggesting that disruption of HFA in the epilepsy network may limit to 60 mm away from SOZ.

In further detail referring now to FIGS. 11A and 11B, shown are exemplary graphs of within-frequency directional information flow as a function of distance between electrodes. As shown in FIG. 11A, the left panel indicates the mean SOZ to non-SOZ information flow of all electrode pairs in distance-range quartiles. The right panel indicates the mean non-SOZ to SOZ information flow of all electrode pairs in distance-range quartiles. FIG. 11B, shows the statistical difference between SOZ to non-SOZ DTF and non-SOZ to SOZ information flow in distance-range quartiles. As shown in FIG. 11B, the significant area at p=0.05 level after FDR correction is marked in shadow.

Referring now to FIGS. 13A-13C, shown are exemplary graphs of a within-frequency directional information flow as a function of distance between electrodes. FIG. 13A, shows the mean information flow of all electrode pairs within SOZ in distance-range quartiles. FIG. 13B shows the mean information flow of all electrode pairs within non-SOZ in distance-range quartiles. FIG. 13C shows the statistical difference between information flow within SOZ and information flow within non-SOZ in distance-range quartiles. Significant area at p=0.05 level after FDR correction is marked in shadow. Data are shown in mean and standard error.

Contributions of resting-state data in clinical decision-making: Ultimately, this study aims to improve seizure outcome of epilepsy patients, which largely relies on the precise delineation of epileptic networks. Here, it was demonstrated that the approach could predict both SOZ and seizure outcome with about 80% accuracy in a large cohort of 43 drug resistant focal epilepsy patients using 10 minutes of interictal intracranial EEG resting-state data. The interpretations of ictal data have limitations, mainly imposed by accelerated meditation changes during the intracranial study and electrode coverage leading to sampling biases that may affect localization accuracy. Moreover, it is challenging to capture all types of seizures during hospitalization. For example, one study showed that approximately one-third of bilateral temporal lobe epilepsy patients required more than four weeks of recordings to capture bilateral independent seizures. In addition, seizure clusters may provide discordant data that may misdirect interpretation and surgical treatment.

The underlying pathology of seizure generation most likely involves both abnormal brain structures and aberrant connections among these regions, leading to large-scale network disruptions. The aberrant network connection could be studied under resting-state, and many resting-state iEEG studies have shown overwhelmingly enhanced connectivity involving EZ and surrounding regions.

To tackle the problem of severely imbalanced data between SOZ and non-SOZ, a balanced random forest model was introduced by optimizing the cost function and the sampling technique. The results, using a network connectivity approach, demonstrated enhanced performance of localizing SOZ with an AUC of 0.94 and an accuracy of 0.85. The E:I ratio was examined by computing the 1/f power slope and provided deeper mechanistic insights between the E:I alteration and aberrant connectivity in the resting-state epilepsy network. Furthermore, by utilizing directional connectivity network information, an important advancement to predict seizure outcome with satisfactory accuracy was made. Prospective validation of the findings would pave the way to reducing traditional prolonged seizure recordings, leading to shorter hospitalizations and improved patient care.

In summary, it was found that the higher excitability in non-SOZ versus SOZ regions could be linked to the dominant information flows from non-SOZ to SOZ at multiple frequencies, probably reflecting insufficient excitability to initiate seizure and widespread network inhibition to prevent seizure initiation. Moreover, stronger information flow from non-SOZ to SOZ was found in seizure free outcome patients compared to non-seizure free outcome patients. In combination with the balanced random forest machine learning model and resting-state connectivity, localization of SOZ and seizure outcome prediction without long-term recordings may supplement traditional interpretation of SEEG and help identify epilepsy treatment targets, thus improving patient care and treatment outcome. The findings are stable and robust under different durations with 5 min, 15 min and 30 min (as shown in FIGS. 13A-C).

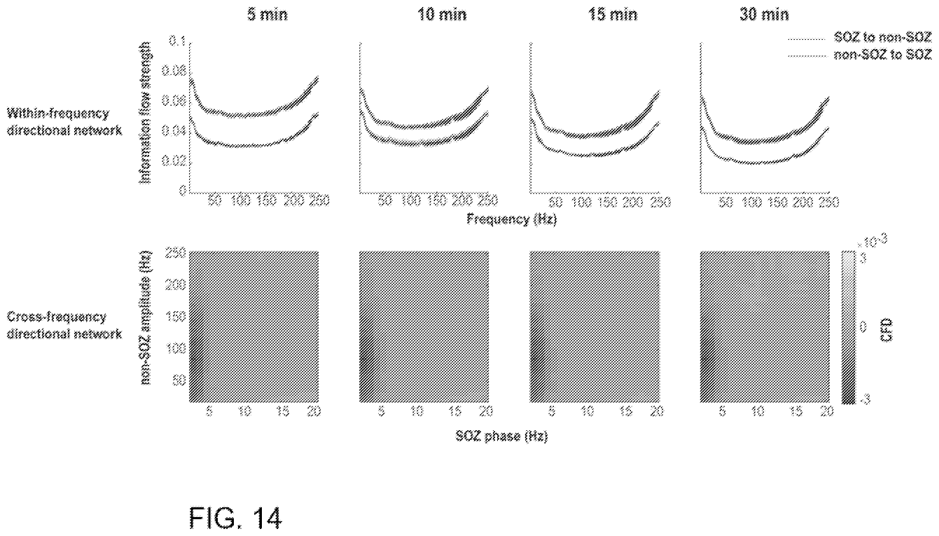

Referring now to FIG. 14, shown is a graph of control analysis of different durations in within-frequency directional network and cross-frequency directional network. As shown in FIG. 14, the overall patterns are similar in 5 min, 10 min, 15 min and 30 min of resting-state.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method for characterizing an epileptic seizure onset zone in a patient with epilepsy, the method comprising:
receiving, by at least one processor, interictal electroencephalographs, optionally including interictal epileptiform discharges, from two or more points in a patient's cerebral cortex, wherein each of the two or more points represent an electrode of a plurality of electrodes;
determining, by the at least one processor, directional information flow between the two or more points based on the interictal encephalographs by quantifying directional information flow value(s) at each electrode of the plurality of electrodes at a plurality of frequencies, wherein the directional information flow value(s) indicate(s) information flow to an electrode from one or more other electrodes and/or from a non-seizure onset zone to a seizure onset zone;
inputting, by the at least one processor, the directional information flow value(s) into a classification model, wherein the classification model is trained to perform a first task and/or a second task, wherein the first task comprises locating the seizure onset zone in the patient's cerebral cortex based on the directional flow value(s), such as the inward directional flow value(s), e.g., a summation of information flow from all other electrodes to an electrode of interest, and quantifying directional information flow values, at an electrode of the plurality of electrodes, and wherein the second task comprises classifying the patient's predicted post-treatment seizure outcome after epilepsy treatment based on the directional information flow from the non-seizure onset zone to the seizure onset zone; and
receiving, by the at least one processor, an output from the classification model based on inputting the directional information flow value(s) into the classification model, wherein the output indicates the location of the seizure onset zone in the patient's cerebral cortex and/or the patient's predicted post-treatment seizure outcome after epilepsy treatment.

2. The method of claim 1, wherein the classification model is trained to perform the first task and wherein the output from the classification model indicates the location of the seizure onset zone in the patient's cerebral cortex based on the directional information flow value(s), such as the inward directional flow value(s).

3. The method of claim 1, wherein the classification model is trained to perform the second task and wherein the output from the classification model indicates the patient's predicted post-treatment seizure outcome after epilepsy treatment based on the directional information flow value(s).

4. The method of claim 1, wherein the directional informational flow value(s) are determined using a Granger causality analysis (GCA), and wherein the directional information flow value(s) represent a Granger-cause.

5. The method of claim 4, wherein the GCA is performed using a directed transfer function (DTF) or using one of: a partial directed coherence (PDC), and adaptive DTF, an adaptive PDC, and/or a cross-frequency directionality.

6. (canceled)

7. The method of claim 1, further comprising:
inputting 1/f power values into the classification model; and
receiving a second output from the classification model based on inputting the 1/f power values into the classification model, wherein the second output indicates the location of the seizure onset zone in the patient's cerebral cortex and/or the patient's predicted post-treatment seizure outcome after epilepsy treatment.

8. (canceled)

9. The method of claim 1, wherein the patient's predicted post-treatment seizure outcome after epilepsy treatment is classified according to a two-tier scale, an Engel outcome scale, and/or an International League Against Epilepsy (ILAE) outcome scale, wherein the output comprises a value indicating the patient's score according to the two-tier scale, the Engel outcome scale, and/or the ILAE outcome scale, wherein the two-tier scale comprises seizure free and non-seizure free.

10. (canceled)

11. The method of claim 1, wherein the interictal electroencephalographs are in a range of 1 Hz to 1,000 Hz and/or the interictal electroencephalographs range from one second to thirty minutes in duration, including any increment therebetween.

12. (canceled)

13. The method of claim 1, wherein the classification model is a decision tree classification model.

14. The method of claim 1, wherein the decision tree classification model is a Random Forest model, and optionally a Balanced Random Forest model.

15. (canceled)

16. The method of claim 13, wherein the decision tree classification model is balanced by minimizing an overall cost by assigning a high cost to a misclassification of a minority class and/or either over-sampling the minority class or down-sampling a majority class, or both.

17. The method of claim 13, wherein the decision tree classification model is trained using a synthetic minority over-sampling (SMOTE) procedure, wherein the minority class of data is over-sampled, and wherein the majority class of data is under-sampled.

18. The method of claim 1, further comprising:
determining a treatment plan for the patient based on the output from the classification model;
communicating data associated with the treatment plan to a user device; and
displaying the data associated with the treatment plan via a graphical user interface on the user device.

19. (canceled)

20. The method of claim 19, further comprising:
performing epilepsy surgery or laser ablation or neuromodulation on the patient when the treatment plan recommends epilepsy surgery or laser ablation or neuromodulation.

21. The method of claim 1, further comprising:
categorizing the patient into a patient sub-population, wherein the patient sub-population comprises: temporal lobe epilepsy, frontal lobe epilepsy, parietal lobe epilepsy, occipital lobe epilepsy, adult epilepsy, and pediatric epilepsy; and
generating the classification model based on the patient's patient sub-population.

22. The method of claim 1, further comprising:
integrating brain imaging data with the output of the classification model, wherein the brain imaging data comprises brain images.

23. The method of claim 1, further comprising:
filtering the interictal electroencephalographs using a band-pass filer between 0.5 Hz and 1,000 Hz; and
performing artifact rejection on the interictal electroencephalographs.

24. The method of claim 1, further comprising:
communicating data associated with the patient's predicted post-treatment seizure outcome after epilepsy treatment to a user device; and
displaying the data associated with the patient's predicted post-treatment seizure outcome after epilepsy treatment via a graphical user interface on the user device.

25. A system for characterizing an epileptic seizure onset zone in a patient with epilepsy, the system comprising at least one processor programmed or configured to:
receive interictal electroencephalographs, optionally including interictal epileptiform discharges, from two or more points in a patient's cerebral cortex, wherein each of the two or more points represent an electrode of a plurality of electrodes;
determine directional information flow between the two or more points based on the interictal encephalographs by quantifying the directional information flow value(s) at each electrode of the plurality of electrodes at a plurality of frequencies, wherein the directional information flow value(s) indicate(s) information flow to an electrode from one or more other electrodes and/or from a non-seizure onset zone to a seizure onset zone;
input the directional information flow value(s) into a classification model, wherein the classification model is trained to perform a first task and/or a second task, wherein the first task comprises locating the seizure onset zone in the patient's cerebral cortex based on the directional flow value(s), such as the inward directional flow value(s), e.g., a summation of information flow from all other electrodes to an electrode of interest, and quantifying directional information flow values, at an electrode of the plurality of electrodes, and wherein the second task comprises classifying the patient's predicted post-treatment seizure outcome after epilepsy treatment based on the directional information flow value(s) from the non-seizure onset zone to the seizure onset zone; and
receive an output from the classification model based on inputting the directional information flow value(s) into the classification model, wherein the output indicates the location of the seizure onset zone in the patient's cerebral cortex and/or the patient's predicted post-treatment seizure outcome after epilepsy treatment.

26-47. (canceled)

48. A computer program product for characterizing an epileptic seizure onset zone in a patient with epilepsy, the computer program product comprising at least one non-transitory computer-readable medium including one or more instructions that, when executed by at least one processor, cause the at least one processor to:
receive interictal electroencephalographs, optionally including interictal epileptiform discharges, from two or more points in a patient's cerebral cortex, wherein each of the two or more points represent an electrode of a plurality of electrodes;
determine directional information flow between the two or more points based on the interictal encephalographs by quantifying the directional information flow value(s), at each electrode of the plurality of electrodes at a plurality of frequencies, wherein the directional information flow value(s) indicate(s) information flow to an electrode from one or more other electrodes and/or from a non-seizure onset zone to a seizure onset zone;
input the directional information flow value(s) into a classification model, wherein the classification model is trained to perform a first task and/or a second task, wherein the first task comprises locating the seizure onset zone in the patient's cerebral cortex based on the directional information flow value(s), such as the inward directional flow value(s), e.g., a summation of information flow from all other electrodes to an electrode of interest, and quantifying directional information flow values, at an electrode of the plurality of electrodes, and wherein the second task comprises classifying the patient's predicted post-treatment seizure outcome after epilepsy treatment based on the directional information flow from the non-seizure onset zone to the seizure onset zone; and
receive an output from the classification model based on inputting the directional information flow value(s) into the classification model, wherein the output indicates the location of the seizure onset zone in the patient's cerebral cortex and/or the patient's predicted post-treatment seizure outcome after epilepsy treatment.

49-70. (canceled)
